(19) United States
(12) Patent Application Publication
(10) Pub. No.: US 2007/0104667 A1
(43) Pub. Date: May 10, 2007

(54) Cosmetic composition comprising a cellulose or a liposoluble modified cellulose derivative

(76) Inventors: Jean Mondet, Aulnaysous-Bois (FR); Xavier Blin, Paris (FR); Ivan Rodriguez, Cauffry (FR); Pascal Arnaud, I'Hya les Roses (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/563,819

(22) PCT Filed: May 14, 2004
(86) PCT No.: PCT/FR04/01202

§ 371 Date: Jan. 16, 2007

Related U.S. Application Data
(60) Provisional application No. 60/495,726, filed on Aug. 18, 2003.

(30) Foreign Application Priority Data
Jul. 7, 2003 (FR) 03/50299

(51) Int. Cl. A61K 8/73

(52) U.S. Cl. 424/70.7; 424/70.13

(57) ABSTRACT

The invention relates to a cosmetic composition for making up the skin, the lips, the eyelashes or the nails, comprising, in a cosmetically acceptable medium, a liquid fatty phase and a liposoluble modified cellulose or cellulose derivative, the said modified cellulose or the said modified cellulose derivative comprising free hydroxyl functions totally or partially replaced with hydrophobic groups chosen from the radicals of formula —OYR, in which: R represents a group chosen from: A) hydrocarbon-based groups containing linear or branched, saturated or unsaturated chains, or saturated or unsaturated rings, containing from 8 to 50 carbon atoms for the modified cellulose or from 4 to 50 carbon atoms for the modified cellulose derivative, the said groups possibly comprising in their chains one or more aromatic groups and/or one or more hetero atoms chosen from O, N, P, Si and S; the said groups possibly being fluorinated or perfluorinated; B) groups of polymeric nature chosen from polyolefins, hydrogenated or non-hydrogenated polydienes and lipophilic polycondensates, and mixtures thereof, Y represents a single bond or a divalent bonding group.

The present invention relates to a cosmetic composition for making up or caring for keratin materials such as the skin, including the scalp, of either the human face or body, the lips and the nails, and keratin fibres such as the eyelashes, the eyebrows and the hair, comprising a cosmetically acceptable medium containing a liposoluble modified cellulose or a liposoluble modified cellulose derivative.

The composition may be a makeup composition such as a loose or compact powder, a foundation, a makeup rouge, an eye shadow, a concealer product, a blusher, a lipstick, a lip balm, a lip gloss, a lip pencil, an eye pencil, a mascara, an eyeliner, a nail varnish, a body makeup product or a skin colouring product.

The term “makeup composition” means a composition intended to deposit a coloured film onto the skin. Makeup compositions preferably comprise a dyestuff such as a dye or a pigment.

The known makeup compositions often have poor resistance over time and in particular poor resistance of the colour. This poor resistance is characterized by a modification of the colour (colour change or fading) generally following an interaction with the sebum and/or sweat secreted by the skin in the case of foundations and powders, or an interaction with saliva in the case of lipsticks. This obliges the user to reapply makeup very regularly, which may represent a loss of time.

Lip and skin makeup compositions referred to as being “transfer-free” are compositions that have the advantage of forming a deposit that does not become deposited, at least partly, onto the supports with which they come into contact (glass, clothing, cigarette or fabrics).

An improvement in the resistance and in the transfer resistance, in particular of lipsticks, may be obtained by combining a volatile oil with a film-forming polymer, for instance silicone resins. However, these compositions have the drawback of leaving on the skin and the lips, after evaporation of the volatile silicone oils, a film that becomes uncomfortable over time (sensation of drying-out and of tautness), which puts a certain number of women off this type of lipstick. Furthermore, the resistance properties obtained are below consumers' expectations.

In the particular case of a composition for coating keratin fibres, especially the eyelashes (mascara), a person skilled in the art is confronted with the problems of resistance to water, to rubbing or to sweat or sebum, which are characterized, for example, by the formation of rings under the eyes and/or the formation of grains due to the erosion of the mascara film.

It is thus sought to obtain a composition that allows the production, after application to the eyelashes, of a makeup film that shows good resistance, in particular to water, for example during bathing or showering, to rubbing, especially with the fingers, and/or to tears, sweat or sebum.

There is thus still a need for a cosmetic product that leads to a deposit on keratin materials, in particular a makeup, which, at the same time, has good resistance, transfer resistance and texture, is easy to apply and gives a uniform deposit.

The composition of the invention may in particular be a makeup product for the human body, lips, nails or keratin fibres such as the eyelashes, in particular having non-therapeutic care and/or treatment properties. It is especially a lipstick or a lip gloss, a makeup rouge, an eye shadow, a tattoo product, a makeup or care product for keratin fibres, especially for the eyelashes (mascara) to be applied as a top coat or a base coat, an eyeliner, a nail varnish, an artificial tanning product for the skin, a hair colouring product or a haircare product.

In particular, the composition according to the invention is a skin makeup composition such as a foundation, an eye shadow, a makeup rouge, a concealer product, a body makeup product, a lip makeup product such as a lipstick or a lip gloss, an eyelash makeup product or mascara, or a nail makeup product or nail varnish.

The inventors have found, surprisingly, that the use of cellulose or cellulose derivatives modified with hydrophobic groups so as to make them liposoluble allows the production of cosmetic compositions that lead to deposits on keratin materials which show good resistance.

One subject of the invention is, more specifically, a cosmetic composition, in particular for making up the skin, the lips, the eyelashes or the nails, comprising, in a cosmetically acceptable medium, a liquid fatty phase and a liposoluble modified cellulose or cellulose derivative, the said modified cellulose or the said modified cellulose derivative comprising free hydroxyl functions totally or partially replaced with hydrophobic groups chosen from the radicals of formula
—OYR, in which:
R represents a group chosen from:
   A) hydrocarbon-based groups containing linear or branched, saturated or unsaturated chains, or saturated or unsaturated rings, containing from 8 to 50 carbon atoms for the modified cellulose or from 4 to 50 carbon atoms for the modified cellulose derivative,
   the said groups possibly comprising in their chains one or more aromatic groups and/or one or more hetero atoms chosen from O, N, P, Si and S; the said groups possibly being fluorinated or perfluorinated;
   B) groups of polymeric nature chosen from polyolefins, hydrogenated or non-hydrogenated polydienes and lipophilic polycondensates, and mixtures thereof,
Y represents a single bond or a divalent bonding group.

The term “liposoluble” means soluble at a concentration of at least 1% by weight relative to the total weight of the composition, in the oil forming the weight majority of the liquid fatty phase, at room temperature (25° C.) and atmospheric pressure (10⁵ Pa).

The term “hydrocarbon-based” means consisting essentially of carbon and hydrogen atoms.

The invention also relates to a cosmetic process for caring for or making up the skin, the lips, the eyelashes or the nails, which consists in applying the composition as defined above to the skin, the lips, the eyelashes or the nails.

Preferably, the composition according to the invention is a leave-in composition.

The invention also relates to the cosmetic use of the composition defined above for improving the resistance and/or the transfer resistance of the makeup on the keratin materials.

In particular, in the case of a composition for coating the eyelashes, or mascara, such a composition allows the production, after application to the eyelashes, of a makeup film that shows good resistance, in particular to water, for example during bathing or showering, to rubbing, especially with the fingers, and/or to tears, sweat or sebum.

Finally, a subject of the invention is the use of a liposoluble modified cellulose or cellulose derivative, the said modified cellulose or the said modified cellulose derivative comprising free hydroxyl functions totally or partially replaced with hydrophobic groups chosen from the radicals of formula —OYR, in which:
R represents a group chosen from:
   A) hydrocarbon-based groups containing linear or branched, saturated or unsaturated chains, or saturated or unsaturated rings, containing from 8 to 50 carbon atoms for the modified cellulose or from 4 to 50 carbon atoms for the modified cellulose derivative,
   the said groups possibly comprising in their chains one or more aromatic groups and/or one or more hetero atoms chosen from O, N, P, Si and S; the said groups possibly being fluorinated or perfluorinated;
   B) groups of polymeric nature chosen from polyolefins, hydrogenated or non-hydrogenated polydienes and lipophilic polycondensates, and mixtures thereof,
Y represents a single bond or a divalent bonding group, to obtain a composition that has a good texture, that is easy to apply and that gives on the skin, the lips or keratin fibres a deposit that shows good resistance and/or that does not transfer.

It is pointed out that, in the text hereinabove and hereinbelow, the term “single bond” means a single covalent bond forming a bridge between the oxygen and the group R. In this case, the group —OYR corresponds to a group —OR.

In the text hereinabove and hereinbelow, the term “divalent bonding group” means an organic spacer group forming a bridge between the oxygen atom and the group R, the said bonding groups possibly being chosen from the groups —(C═O)—, —(C═O)O—, —SO₂—, —CO—NH—, —CO—NR′— and —Si(R₃)₂—, the groups R₃, which may be identical or different, being a linear or branched hydrocarbon-based group containing from 1 to 500 carbon atoms, or a cyclic hydrocarbon-based group containing from 3 to 500 carbon atoms, the said group being saturated or unsaturated and possibly comprising one or more hetero atoms chosen from O, N, S, Si and/or P, and R′ denoting an alkyl radical containing from 1 to 4 carbon atoms.

Preferably, R₃ represents an alkyl group containing from 1 to 10 carbon atoms.

The liquid fatty phase of the composition according to the invention comprises at least one (i.e. one or more) mutually compatible fatty substances that are liquid at room temperature (25° C.) and atmospheric pressure (10⁵ Pa).

I) Liposoluble Modified Cellulose or Cellulose Derivative

For the purposes of the present description, the terms “cellulose” and “cellulose derivative” mean the cellulose or cellulose derivative before partial or total substitution of their free hydroxyl functions with hydrophobic groups of formula —OYR to make them liposoluble.

For the purposes of the present description, the terms “modified cellulose” and “modified cellulose derivative” mean the cellulose or the cellulose derivative obtained after partial or total substitution of their free hydroxyl functions with hydrophobic groups of formula —OYR, this modified cellulose or this modified cellulose derivative being liposoluble.

According to one embodiment, a cellulose derivative modified by partial or total substitution of its free hydroxyl functions with hydrophobic groups of formula —OYR is used in the composition. The cellulose derivative (unmodified) is chosen from cellulose esters and ethers.

Cellulose Esters

It is pointed out that the term “cellulose ester”, before modification with the groups —O—Y—R, means, in the text hereinabove and hereinbelow, a polymer consisting of an α(1-4) sequence of partially or totally esterified anhydroglucose rings, the esterification being obtained by reaction of all or only some of the free hydroxyl functions of the said anhydroglucose rings with a linear or branched carboxylic acid or carboxylic acid derivative (acid chloride or acid anhydride) containing from 1 to 4 carbon atoms.

Preferably, the cellulose ester results from the reaction of some of the free hydroxyl functions of the said rings with a carboxylic acid containing from 1 to 4 carbon atoms.

Advantageously, the cellulose esters are chosen from cellulose acetates, propionates, butyrates, isobutyrates, acetobutyrates and acetopropionates, and mixtures thereof.

These cellulose esters may have a weight-average molecular mass ranging from 3000 to 1 000 000, preferably from 10 000 to 500 000 and preferably from 15 000 to 300 000.

According to one preferred embodiment, a subject of the invention is a cosmetic composition, in particular for making up the skin, the lips or the eyelashes, comprising, in a cosmetically acceptable medium, a liquid fatty phase and a liposoluble modified cellulose ester, the said modified cellulose ester comprising free hydroxyl functions totally or partially replaced with hydrophobic groups chosen from the radicals of formula —OYR, in which:
R represents a group chosen from:
   A) hydrocarbon-based groups containing linear or branched, saturated or unsaturated chains, or saturated or unsaturated rings, containing from 4 to 50 carbon atoms,
   the said groups possibly comprising in their chains one or more aromatic groups and/or one or more hetero atoms chosen from O, N, P, Si and S; the said groups possibly being fluorinated or perfluorinated;
   B) groups of polymeric nature chosen from polyolefins, hydrogenated or non-hydrogenated polydienes and lipophilic polycondensates, and mixtures thereof,
Y represents a single bond or a divalent bonding group.
   Cellulose Ethers:

In the text hereinabove and hereinbelow, the term “cellulose ether” means a polymer consisting of an α(1-4) sequence of partially etherified anhydroglucose rings, some of the free hydroxyl functions of the said rings being substituted with a radical —OR, R preferably being a linear or branched alkyl radical containing from 1 to 4 carbon atoms.

The cellulose ethers are thus preferably chosen from cellulose alkyl ethers with an alkyl group containing from 1 to 4 carbon atoms, such as cellulose methyl, propyl, isopropyl, butyl and isobutyl ethers.

These cellulose ethers may have a weight-average molecular mass ranging from 3000 to 1 000 000, preferably from 10 000 to 500 000 and preferably from 15 000 to 300 000.

Thus, the liposoluble modified cellulose derivative (final) of the composition according to the invention comprises hydroxyl groups substituted simultaneously:
a) with groups preferably chosen from carboxylic acid esters containing from 1 to 4 carbon atoms or alkyl ethers with an alkyl group containing from 1 to 4 carbon atoms, and
b) with “hydrophobic” groups chosen from the radicals of formula —OYR as defined above.

The modified cellulose and cellulose derivative are liposoluble, i.e. soluble at a concentration of at least 1% by weight relative to the total weight of the composition, in the oil forming the weight majority of the liquid fatty phase, at room temperature (25° C.) and atmospheric pressure (10⁵ Pa).

Preferably, the modified cellulose or the modified cellulose derivative is totally soluble in the oil forming the weight majority of the liquid fatty phase, i.e. completely dissolved at a concentration of greater than or equal to 1% by weight, at room temperature and atmospheric pressure, in the said majority oil.

When the liquid fatty phase comprises a mixture of oils, in the case of two or more oils present in identical mass proportions, the modified cellulose or the modified cellulose derivative is soluble in at least one of them.

The value of the modified cellulose or modified cellulose derivative used in the context of the invention lies in the fact that they show good solubility in the liquid fatty phase of the composition according to the invention as defined above and afford the compositions comprising them improved resistance properties (resistance to water, saliva or sweat), in particular when the liquid fatty phase predominantly comprises a volatile oil. These compounds also allow “transfer-resistant” compositions to be obtained, in particular when the liquid fatty phase predominantly comprises a volatile oil.

This liposoluble modified cellulose or cellulose derivative according to the present invention is preferably a film-forming polymer.

The term “film-forming polymer” means a polymer capable of forming, by itself or in the presence of an auxiliary film-forming agent, a macroscopically continuous film that adheres to keratin materials, preferably a cohesive film and better still a film whose cohesion and mechanical properties are such that the said film can be isolated and handled in isolation, for example when the said film is made by pouring onto a non-stick surface, for instance a Teflon-coated or silicone-coated surface.

The modified cellulose or cellulose derivatives of the composition according to the invention may also act as thickener or even as gelling agent for the liquid fatty phase of the composition.

The cellulose or cellulose derivative is modified with hydrocarbon-based (non-polymeric) or polymeric groups —O—Y—R as described below.

A/ Hydrocarbon-Based Groups R

According to one preferred embodiment of the invention, the groups R are non-polymeric hydrocarbon-based groups corresponding to the definition A) given above, i.e. they may be a saturated or unsaturated hydrocarbon-based group, containing a linear or branched, cyclic or non-cyclic chain comprising:
from 4 to 50 atoms when the composition comprises a cellulose derivative,
from 8 to 50 carbon atoms when the composition comprises a modified cellulose,
   the said hydrocarbon-based group possibly comprising in its chain one or more aromatic groups and/or one or more hetero atoms chosen from O, N, P, Si and S,
these groups possibly being fluorinated or perfluorinated.

It is pointed out that these groups are of non-polymeric nature, i.e. they do not result from the polymerization or polycondensation of several monomers.

According to one embodiment, the groups R are chosen from:
linear-chain hydrocarbon-based groups containing from 8 to 25 carbon atoms for the modified cellulose and linear-chain hydrocarbon-based groups containing from 4 to 25 carbon atoms for the modified cellulose derivatives, in particular saturated linear hydrocarbon-based groups containing from 8 to 11 carbon atoms and linear hydrocarbon-based groups with at least one unsaturation, containing from 8 to 22 carbon atoms.
   Mention may be made especially of saturated linear alkyl groups such as n-butyl, n-pentyl, n-hexyl, n-heptyl, n-octyl, n-nonyl, n-decyl and n-undecyl, and mixtures thereof;
saturated branched-chain hydrocarbon-based groups containing from 8 to 50 carbon atoms for the modified cellulose and saturated branched-chain hydrocarbon-based groups containing from 4 to 50 carbon atoms and preferably from 8 to 50 carbon atoms for the modified cellulose derivatives. Mention may be made in particular of branched alkyl groups containing from 8 to 40 carbon atoms. Mention may be made especially of isobutyl, tert-butyl, isopentyl, tert-hexyl, 2-ethylhexyl, tert-octyl, isononyl, isodecyl, neodecyl, isododecyl, isohexadecyl and isostearyl groups;
cyclic hydrocarbon-based groups containing from 8 to 50 carbon atoms and preferably from 8 to 20 carbon atoms for the modified cellulose and cyclic hydrocarbon-based groups containing from 6 to 50 carbon atoms and preferably from 6 to 20 carbon atoms for the modified cellulose derivatives. Mention may be made especially of cyclohexyl, isobornyl, adamantyl and norbornyl groups;
branched and/or cyclic hydrocarbon-based groups derived from unsaturated fatty acid derivatives containing from 12 to 24 carbon atoms, such as alkyl ketene dimers,
and combinations thereof.
   B/ Polymeric Groups R

According to one embodiment of the invention, the groups R may be groups of polymeric nature chosen from polyolefins, polydienes, lipophilic polycondensates such as polyesters, polyamides, polyurethanes, polyureas, copolymers (urea/urethane) and polyethers, and mixtures thereof.

These groups are chosen from lipophilic and preferably liposoluble hydrophobic groups.

1) Polyolefins

Examples of polyolefins that may be mentioned include the polymers obtained by homopolymerization or copolymerization of monomers chosen from: α-olefins containing, for example, from 2 to 20 carbon atoms, in particular α-olefin copolymers, the monomers of which give crystalline homopolymers; and branched α-olefin homopolymers or copolymers. Mention may be made in particular of the isobutylene homopolymer and copolymers between ethylene (or propylene) and longer-chain α-olefins such as butene, hexene, octene, decene or dodecene.

Mention may also be made of non-crystalline copolymers derived from the copolymerization between the olefins mentioned above and cycloolefins, in particular copolymers between ethylene or propylene and norbornene or norbornene derivatives.

2) Polydienes

The polydienes may be chosen from polydienes resulting from the polymerization of dienes containing, for example, from 4 to 20 carbon atoms, such as butadiene, isoprene or hexadiene, or from polymers resulting from the polymerization of dienes containing, for example, from 4 to 20 carbon atoms with other vinyl monomers such as the α-olefins mentioned above and/or with styrene or substituted styrenes.

Examples of polydienes are polybutadienes and polyisoprene, which are preferably hydrogenated.

3) Lipophilic polycondensates

According to the invention, the lipophilic polycondensates may be chosen from polyesters, polyamides, polyesteramides, polyurethanes, polycarbonates, polyureas, copolymers (urea/urethane) and polyethers, on condition that they are lipophilic.

3.1/ Examples of lipophilic polyesters that may be mentioned include those derived from the polyesterification of at least one polyol such as a diol or a triol with at least one polycarboxylic acid, such as a dicarboxylic acid or a tricarboxylic acid or a dicarboxylic or tricarboxylic acid derivative (such as an acid chloride or an acid anhydride), or a dialkyl ester containing from 1 to 5 carbon atoms.

The polyester is lipophilic insofar as at least the diol, the polyol, the dicarboxylic acid or the tricarboxylic acid is lipophilic.

a) Polyol

The polyol may be chosen in particular from:
aliphatic diols containing long linear or branched chains, for example of C8 to C40.
   Mention will be made in particular of 1,12-dodecanediol and 1,10-decanediol, the latter being sold by the company Cognis under the name Sovermol 110. It is also possible to use diols of structure: 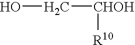
   in which R¹⁰ is an alkyl chain of length C₈-C₃₀, in particular R¹⁰=C₂₄-C₂₈ mixture or R¹⁰=C₂₀-C₂₄ mixture.

These diols are sold by the company Atofina under the trade name Vikinol.

Diol Dimers

The diol dimers are branched diols, generally of C₃₆, often as aliphatic and/or alicyclic mixtures prepared from “fatty acid dimers”, which are compounds of the same structure but having two carboxylic acid end groups (instead of diol end groups).

As indicated in the article by R. Höfer, European Coating Journal, March 2000, pages 26-37, the conversion of the fatty acid dimers into diol dimers may be performed:
either by hydrogenation of methyl esters of fatty acid dimers,
or by direct dimerization of oleyl alcohol.

For a definition of the fatty acid dimers, reference may be made to U.S. Pat. No. 3,157,681 and U.S. Pat. No. 5,998,570.

Mention will be made, in particular, of the diol dimers sold by the company Cognis under the trade names Sovermol 908 (containing 97.5% pure dimer) and Sovermol 650 NS (containing 68% pure dimer).

Polydienes and Polyolefins, Preferably Hydrogenated, Containing Hydroxyl End Groups

These polymers containing hydroxyl end groups are defined, for example, in patent FR 2 782 723. They are preferably chosen from the group comprising butadiene, isoprene and 1,3-pentadiene homopolymers and copolymers. They are oligomers of M_{W}<7000, preferably between 1000 and 5000, with a hydroxyl functionality (at the end groups) of between 1.8 and 3 and preferably in the region of 2.

These polymers are preferably used hydrogenated.

Mention will be made in particular of the hydroxylated polybutadienes sold by Atofina under the brand names Poly BD R-45HT and Poly BD R-20LM, which will preferably be used hydrogenated.

It is also possible to use polyolefin homopolymers or copolymers containing α,ω-hydroxylated end groups, such as:
polyisobutylene oligomers containing α,ω-hydroxyl end groups;
the copolymers sold by the company Mitsubishi under the brand name Polytail, with, in particular, those of structure: 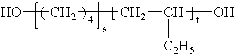
   diols and triols with a long C10 to C50 aliphatic chain to provide affinity (or solubility) in hydrocarbon-based oils.

Among these diols and triols, mention may be made of:
natural or synthetic oils bearing from 2 to 3 hydroxyl groups, the preferred oils being, of course, oils bearing two hydroxyl groups per chain, for instance the monoglycerides of structure: 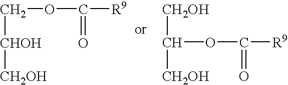
R⁹ being a linear or branched, saturated or unsaturated aliphatic chain containing from 6 to 50 carbon atoms.

Mention will be made, for example, of glyceryl monostearate. Such glycerol monoesters correspond to the general formula:

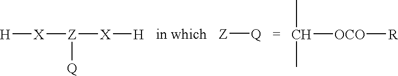

It is also possible to use an oil comprising three hydroxyl groups per chain, for instance hydrogenated or non-hydrogenated castor oil, avoiding crosslinking of the polymer (to maintain good solubility thereof).
Polyols in the case where the lipophilicity of the polymer is provided by the polycarboxylic acid, such as ethylene glycol, propylene glycol, diethylene glycol, neopentyl glycol, 1,4-butanediol, furandimethanol, cyclohexanedimethanol, glycerol, trimethylolpropane and pentaerythritol, and mixtures thereof.
   b) Dicarboxylic and Polycarboxylic Acids:

They are preferably chosen from linear, branched or cyclic dicarboxylic acids, preferably containing from 4 to 60 carbon atoms, or derivatives thereof: anhydrides, acid dichloride, diesters derived from the reaction of a C1-C6 alcohol and in particular those comprising:
a linear or branched C₃-C₅₀ alkyl or alkenyl chain, which may comprise one or more conjugated or non-conjugated unsaturations, which may comprise saturated or unsaturated rings, and may be interrupted with one or more hetero atoms of the type —O—, —S— or —N—;
a C₈-C₃₀ cycloalkyl chain (including the two atoms of the acid groups or acid derivatives), optionally substituted with one or more C₁-C₁₀ alkyl groups;
a C₈-C₃₀ aromatic chain (including the two carbon atoms of the acid groups or acid derivatives), optionally substituted with one or more C₁-C₁₀ alkyl groups.

Aliphatic dicarboxylic acids and derivatives thereof (C1-6 anhydride, acid chloride or ester) are preferred, and in particular those of formula: 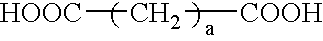
in which a is an integer from 2 to 20; for instance adipic acid, glutaric acid, succinic acid, pimelic acid, azeleic acid, sebacic acid and suberic acid, and derivatives thereof.

Itaconic acid and maleic acid may also be mentioned as aliphatic diacids.

Finally, a second class of aliphatic diacids and derivatives that are particularly preferred are fatty acid dimers, which are branched diacids, generally of C₃₆, often as aliphatic and/or alicyclic mixtures. These fatty acid dimers are defined in particular in U.S. Pat. No. 3,157,681.

Among the aromatic diacids and derivatives, mention may be made of:
the diacids of formula: 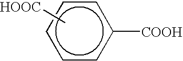
in which the second acid group COOH is in the ortho, meta or para position, the benzene ring optionally being substituted, in particular with one or more alkyl groups, preferably of C₁-C₆;
anthracenedicarboxylic, 1,8-naphthalenedicarboxylic, 1,4-naphthalenedicarboxylic and biphenyldicarboxylic acids, optionally substituted, in particular with one or more alkyl groups, preferably of C₁-C₆;
dichloride and diester derivatives, in particular the C₁-C₆ alkyl diesters, such as methyl and ethyl, of these acids; and
mixtures thereof.

Lipophilic polyesters that may also be mentioned include branched polyesters with a long C10-40 alkyl chain bearing at least two reactive groups, such as poly(12-hydroxystearate) with hydroxyl end groups.

The preparation of poly(12-hydroxystearate) with hydroxyl end groups is described in the article by Radhakrishnan P. N., European Polymer Journal, 35, 2185-92 (1999) and in Indian patent 14 3864. It is obtained via the self-condensation of 12-hydroxystearic acid, followed by reaction with a polyol to consume the residual acid groups. The structure of the oligomer is then as follows: 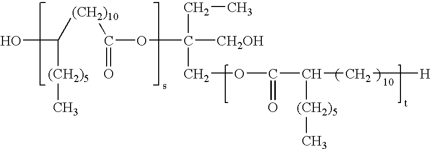
with t such that M_{W}≈2000
3.2/ Polyesteramides

Examples of polyesteramides that may be mentioned include those obtained by reacting amino alcohols such as ethanolamine with the diacids and polyacids mentioned in 3.1/ part b).

3.3/ Polyamides

Examples of polyamides that may be mentioned include those obtained by condensation between an aliphatic, cycloaliphatic or aromatic dicarboxylic acid (or C₁-C₄ ester derivative) containing from 3 to 50 carbon atoms and a linear or branched aliphatic, cycloaliphatic or aromatic diamine containing from 2 to 50 carbon atoms, the diacids possibly chosen from the diacids mentioned above with, in addition, fatty acid dimers (derived from the condensation between two molecules of unsaturated fatty monoacids), the diamines possibly being chosen from ethylenediamine, 1,2-diaminopropane, 1,3-diaminopropane, 1,4-diaminobutane, 1,2-diamino-2-methylpropane, 1,6-diaminohexane, 1,10-diaminodecane, isophoronediamine, adamantanediamine and 2,6-diaminopyridine.

According to one particularly preferred mode of the invention, it is possible to use diamine dimers of the same structure as the diol dimers mentioned above, but containing two primary amine functions instead of two hydroxyls. They may be obtained from fatty acid dimers, like the diol dimers as indicated in the R. Hoffer article mentioned above.

Finally, it is also possible to use diamines of structure H₂N-D-NH₂, in which D is a linear or branched alkyl chain containing from 8 to 40 carbon atoms.

Examples of such diamines that may be mentioned include:
1,10-diaminodecane and 1,12-diaminododecane,
the diamino oils sold by the company Akzo-Nobel under the names:
   Duomeen C or CD: cocopropylenediamine (distilled or undistilled),
   Duomeen HT: hydrogenated tallowpropylenediamine,
   Duomeen M: C₁₆₋₂₂ alkylpropylenediamine,
   Duomeen O: oleylpropylenediamine,
   Duomeen T: tallowpropylenediamine
      3.4/ Polyurethanes, Polyureas and Polyureas-Urethanes

Examples of polyurethanes, polyureas and polyureas-urethanes that may be mentioned include those obtained by polyaddition between aliphatic, cycloaliphatic and/or aromatic diisocyanates containing from 4 to 100 carbon atoms and preferably from 4 to 30 carbon atoms, such as hexamethylene diisocyanate, isophorone diisocyanate, toluene diisocyanate and diphenylmethane diisocyanate, and diols such as those defined above, or diamines such as those defined above, or diol/diamine mixtures.

Advantageously, the group R as defined above may bear one or more groups capable of establishing a hydrogen bond.

The term “group capable of establishing a hydrogen bond” means a group comprising either a hydrogen atom linked to an electronegative atom, or an electronegative atom. When the group comprises a hydrogen atom linked to an electronegative atom, the hydrogen atom may interact with another electronegative atom borne, for example, by another molecule, such as keratin, to form a hydrogen bond. When the group comprises an electronegative atom, the electronegative atom may interact with a hydrogen atom linked to an electronegative atom borne, for example, by another molecule, such as keratin, to form a hydrogen bond.

Advantageously, these groups capable of establishing a hydrogen bond may be groups chosen from the following groups:
hydroxyl —OH;
carboxylic acid —COOH;
amino-NR₁R₂ with R₁ and R₂ being identical or different;
pyridino of formula: 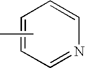
pyrimidino of formula: 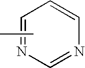
oxazolino corresponding to one of the following formulae: 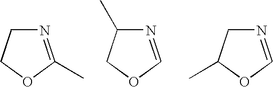
amido of formula —NH—CO—R′ or —CO—NH—R₁;
pyrrolidino corresponding to one of the following formulae: 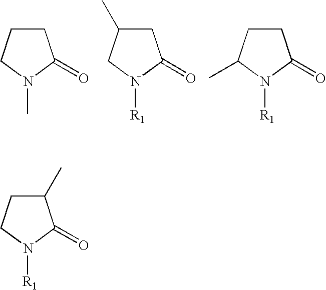
carbamoyl of formula —O—CO—NH—R′ or —NH—CO—O—R′;
thiocarbamoyl of formula —O—CS—NHR₁ or —NH—CS—O—R′;
carbonato —O—CO—O—R′;
ureyl —NR₁—CO—N(R₁)₂, the R₁ being identical or different;
thioureyl —NR₁—CS—N(R₁)₂, the R₁ being identical or different;
oxamido —NR₁—CO—CO—N(R₁)₂ with the R₁ being identical or different;
guanidino —NH—C(═NH)—N(R₁)₂ with the R₁ being identical or different;
biguanidino —NH—C(═NH)—NH—C(═NH)—N(R₁)₂ with the R₁ being identical or different;
sulfonamido —NR₁—S(═O)₂—R′;
with R₁ and R′ representing H or an alkyl group containing from 1 to 4 carbon atoms, R′ representing an alkyl radical containing from 1 to 4 carbon atoms.

It is understood that groups are borne by the chain R either at the end of the chain or laterally to the said chain.

Derivatives bearing at least one group capable of establishing a hydrogen bond are particularly advantageous since they give the compositions containing them very high adhesion properties by virtue of the ability of these groups to establish a hydrogen bond with keratin materials.

The liposoluble modified cellulose or cellulose derivative in accordance with the invention may represent from 0.5% to 50%, preferably from 1% to 45%, better still from 4% to 40% and even better still from 5% to 30% by weight of solids (or active material) relative to the total weight of the composition according to the invention.

According to one embodiment, the liposoluble derivative is present in a content of at least 4% by weight and preferably at least 5% by weight relative to the total weight of the composition.

When the composition comprises a modified cellulose derivative, this derivative may be prepared via various processes within the reach of a person skilled in the art, and in particular according to the following two main synthetic routes:

either starting with cellulose ester or ether derivatives, and these derivatives are reacted with suitable reagents to graft onto the free hydroxyl functions groups of formula —Y—R as defined above (referred to in the description hereinbelow as Route A);

or starting with celluloses already modified with groups —Y—R as defined above, and these celluloses thus modified are reacted with suitable reagents to obtain the esterification or etherification of at least some of the free hydroxyl functions (referred to as route B).

The preferred route for synthesizing the cellulose derivatives of the invention is route A. Only this route will be described in detail in the present patent application.

When the composition comprises a modified cellulose, this cellulose may be prepared via the same synthetic route A above used for the modified cellulose derivative.

According to this synthetic route, the starting reagent is a cellulose, a cellulose ester or a cellulose ether comprising a certain number of free OH functions on which are reacted suitable reagents to give groups —O—Y—R.

If the groups of the divalent bonds —Y— are groups —C—, —O—Y— being an ester group, polymers of the

′′

O invention may also be prepared by partially or totally transesterifying the ester groups of the initial cellulose ester with a fatty acid HOOC—R or with a methyl ester of this fatty acid of formula CH₃OC(O)—R, —R having the above definition and R in particular being a linear, branched or cyclic hydrocarbon-based group.

This transesterification may also be performed with a mixture of fatty acids HOOC—R, or of methyl esters of fatty acids CH₃—O—C(O)—R, the radicals R being different.

In general, the reactions for attaching the groups —Y—R to the starting cellulose, cellulose ethers or cellulose esters differ depending on whether R is a hydrocarbon-based group (non-polymeric) according to definition A) or a polymeric group according to definition B).

I/ When R is a Non-Polymeric Hydrocarbon-Based Group

There are two main types of reaction as indicated above.

1/ When the Starting Reagent is a Cellulose Ester, a Partial or Total Transesterification of the Ester Groups Already Present in the Initial Cellulose Ester is Performed.

This reaction may be performed using a fatty acid R—COOH (R corresponding to the definition given above) or a mixture of fatty acids with radicals R of different nature. This reaction may be performed, according to one advantageous embodiment, by starting not with a fatty acid, but with its methyl ester CH₃—O—C(O)—R, or with a mixture of methyl esters of fatty acids, the reaction being catalysed with sodium methoxide and the methyl alcohol formed during the reaction being distilled off.

In the particular case of these transesterification reactions, the choice of the fatty acid or of the mixture of fatty acids (or of the methyl esters thereof) will relate more particularly to:
carboxylic acids: n-hexanoic acid, n-heptanoic acid, n-octanoic acid, n-nonanoic acid, n-decanoic acid, n-undecanoic acid, in the case where the group R is a linear alkyl radical,
carboxylic acids: tert-butanoic acid, isopentanoic acid, tert-hexanoic acid, isodecanoic acid, isododecanoic acid, neodecanoic acid (=versatic acid), isostearic acid, octyldodecanoic acid, in the case where the group R is a branched alkyl radical.

Needless to say, this route is not applicable if the starting reagent is no longer a cellulose ester, but rather a cellulose or a cellulose ether as defined.

However, via this route, the degree of substitution of the OH functions is limited. If a high degree of substitution is desired, it is preferable to esterify the residual OH groups of the starting cellulose ester directly by using a fatty acid chloride or anhydride.

2/ A Direct Reaction is Performed on the Free OH Functions of the Cellulose or of the Cellulose Ether or Ester Derivative

For the text hereinbelow, in the description of the methods for preparing the modified cellulose or the cellulose derivatives (in particular cellulose ester or ether) modified according to the invention, the following abbreviations will be used:
Cell-OH for the starting unmodified cellulose or unmodified cellulose derivative (cellulose ether or ester), only one OH being taken into account, for the purposes of clarity of the description;
R for the hydrocarbon-based chain to be grafted onto the cellulose, corresponding to the definitions given above.

In the text hereinbelow, various reactions that may be used to graft chains R of non-polymeric nature will be described.

2.1) Etherification

For the etherification, the following reactions may be envisaged, for which reactions the radical Y forming the junction between the chains R and the cellulose or the cellulose ester or ether is a single bond:
a/ reaction with an alkyl halide R—X (X representing a halogen), in basic medium (for example in the presence of aqueous sodium hydroxide solution):
   Cell-OH+R—X→Cell-OR+HX
   with X being a halogen chosen from chlorine, bromine and iodine.
b/ reaction with an epoxide: 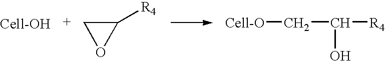
with R₄ representing a chain included in the constitution of the chain R defined above, the said chain R being represented herein by the group —CH₂—(CHOH)—R₄; suitable epoxide reagents may be 1,2-epoxyoctane, 1,2-epoxynonane, 1,2-epoxydecane, 1,2-epoxyneodecane, 1,2-epoxycyclodecane, 1,2-epoxycyclododecane, 1,2-epoxycyclohexane or 1,2-epoxy-3-phenoxypropane.
c/ reaction with an aldehyde in reductive medium (such as triethylsilane in the presence of platinum): 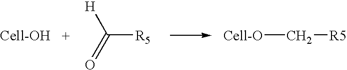
with R₅ representing a chain included in the constitution of the chain R, the said chain R being represented herein by the group —CH₂—R₅.

According to one variant, this reaction may proceed in two steps, the first step being a preliminary reaction of the aldehyde with a diol, such as glycol, to form a cyclic acetal: 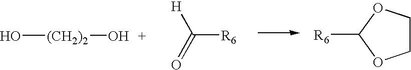
followed by a reaction of the cyclic acetal with the cellulose or the cellulose derivative: 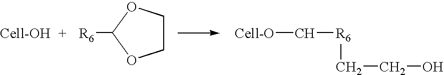
with R₆ representing a chain included in the constitution of the chain R, the said chain R being represented herein by the group —CH—(CH₂—CH₂—OH)—R₆.
d/ Reaction with a mixed ether R—O—R′ with R′ denoting a C₁-C₄ alkyl radical, in acidic medium:
   Cell-OH+R′—O—R→Cell-O—R+R′—OH
e/ addition reaction of the free —OH functions of the cellulose or the cellulose derivative with a double bond, for example a terminal double bond, borne by the radical to be grafted, in the presence of PdCl₂ and HgCl₂:
   Cell-OH+CH₂═CH—R₇→Cell-O—CH₂—CH₂₋R₇
   or
   Cell-OH+Rₐ—CH═CH—Rb→Cell-O—CHRₐ—CH₂—R_{b}
R₇ representing a chain included in the constitution of the chain R, represented herein by the group —CH₂—CH₂—R₇ and Rₐ and R_{b} representing a chain included in the constitution of the chain R, represented herein by the group —CHRₐ—CH₂—R_{b}.

Other etherification reactions may be envisaged, especially those mentioned in the book “Advanced Organic Chemistry”, J. March, John Wiley & son, 1992 edition.

2.2) Esterification.

Examples of esterification reactions that may be mentioned include the following reactions, for which reactions Y represents a CO bonding group:
reaction with a carboxylic acid R—CO₂H:
   Cell-OH+R—CO₂H→Cell-O—CO—R+H₂O
reaction with an acid chloride R—COCl:
   Cell-OH+R—COCl→Cell-O—CO—R+HCl
reaction with an acid anhydride, for example: 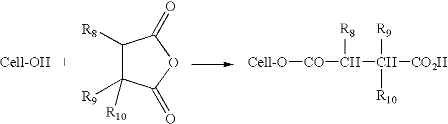
with R₈, R₉ and R₁₀ being such that —(CHR₈)—CR₉R₁₀—CO₂H represents R.

The esterification by reaction with an acid chloride or acid anhydride rather than by reaction with a carboxylic acid is preferred.

Suitable esterification reagents may be: octanoic acid, 2-ethylhexanoic acid, nonanoic acid, decanoic acid, neodecanoic acid, undecanoic acid, dodecanoic acid, isononanoic acid, isodecanoic acid, isododecanoic acid, isostearic acid.

Among the acid chlorides that may be mentioned are: neodecanoyl chloride, undecanoyl chloride, decanoyl chloride, dodecanoyl chloride and isononanoyl chloride.

Among the acid anhydrides, mention will be made most particularly of alkylsuccinyl anhydrides, and in particular: isooctadecenylsuccinic anhydride, 1-octenylsuccinic anhydride, 1-nonenylsuccinic anhydride.
the esterification may also be performed by reacting the residual —OH groups of the cellulose derivative with reactive alkyl ketene dimers (AKD) containing a monofunctional cyclic ketene group.

AKDs are obtained by reaction starting with a mixture of unsaturated fatty acids containing from 14 to 22 carbon atoms, and having the following formula: 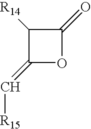
with R₁₄ and R₁₅, which may be identical or different, being a C₈₋₂₀ alkyl chain such that the sum of the number of carbon atoms on the chains R₁₄ and R₁₅ ranges from 30 to 36 atoms.

By way of example, R₁₄=C₁₄H₂₉ and R₁₅=C₁₆H₃₃.

The esterification reaction is then the following with Cell-OH

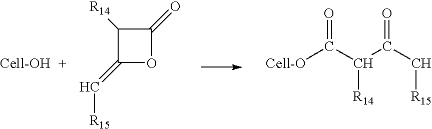

These compounds are described in the following documents: Nahm, S. H., J of Wood Chemistry and Technology, 6(1), 89-112 (1986); Bottorff, K. J., Tappi Journal, 77(4), 105-116 (1994) and Zhou, Y. J., Paper Technology, July 1991, p. 19-22.

In general, the preparation of cellulose or cellulose derivatives modified with liposoluble groups —O—Y—R is advantageously performed via the esterification reactions described above.

2.3) Transesterification with a Carbonate

The following reaction with a carbonate R′—O—CO—O—R may be envisaged, for which reaction Y represents a —CO—O— bonding group:
Cell-OH+R′—O—CO—O—R→Cell-O—CO—O—R+R′—OH
2.4) Esterification with a Sulfonyl Chloride

As examples for the esterification reactions with a sulfonic acid or a sulfonyl chloride, for which reactions Y represents an —SO₂— bonding group, the following reaction may be envisaged:
Cell-OH+Cl—SO₂—R→Cell-O—SO₂—R+HCl
2.5) Reaction with an Isocyanate

As examples for the reactions for formation of carbamate bonds, for which reactions Y represents a —CO—NH— bonding group, the following reaction with an isocyanate OCN—R may be envisaged:
Cell-OH+OCN—R→Cell-O—CO—NHR

Suitable isocyanate reagents may be butyl isocyanate, isobutyl isocyanate, pentyl isocyanate, hexyl isocyanate, heptyl isocyanate, octyl isocyanate, 2-ethylhexyl isocyanate, nonyl isocyanate, decyl isocyanate, undecyl isocyanate, dodecyl isocyanate or phenyl isocyanate.

This reaction is particularly preferred.

2.6) Reaction with an Alkoxysilane

As examples for the reactions with an alkoxysilane, for which reactions Y represents an —Si(R₁₂)₂— bonding group, the following reaction may be envisaged: 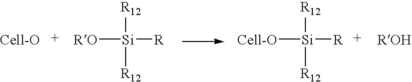
with the R₁₂, which may be identical or different, possibly being a linear, branched or cyclic, saturated or unsaturated hydrocarbon-based chain of 1 to 500 carbon atoms, which may comprise one or more O, N, S, Si and/or P atoms, preferably of 1 to 10 carbon atoms, the groups R₁₂ thus having the same definition as the groups R₃ defined above.
II/ When R is a Polymeric Group

When R is a group of polymeric origin as described above, the grafting reaction may be performed according to the following scheme:
Cell-OH+X₁-POL→Cell-O—Y-POL
with POL representing the polymer whose sequence corresponds to the definition of the group of polymeric nature R given above, X₁ representing a function borne by the polymer, the said function being reactive towards the hydroxyls of the initial cellulose or of the initial cellulose derivative (cellulose ester or ether) and Y corresponding to the same definition as that given above and resulting from the reaction of —OH with X₁.

In the same respect as for the grafting of hydrocarbon-based chains of non-polymeric nature, the reactive functions Xi that are reactive towards the free hydroxyl functions of the initial cellulose derivative may be chosen from epoxide, aldehyde, acetal, halogen (chlorine, bromine or iodine), ethylenic, carboxylic acid or derivative (chloride, anhydride or C₁-C₄ alkyl ester), carbonate, sulfonic acid, sulfonyl chloride, isocyanate and monoalkoxysilane functions.

The starting polymers POL-X₁ need to be synthesized, apart from those for which X₁ is a reactive double bond of vinyl type, many of which are commercially available. The polymers POL-X₁ may be synthesized, for example, from a polymer comprising a reactive function other than X₁, which is converted via standard reactions into a suitable X₁.

An example that may be mentioned is the following reaction:

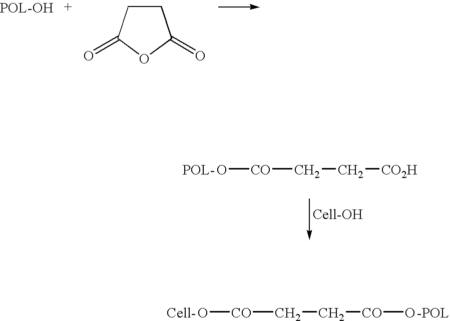

As regards polycondensates containing a reactive end group X₁, their preparation, in particular as regards polyesters and polyamides, does not require any particular arrangement for the introduction of the reactive group, insofar as such a group is already present at the end of the chain.

For example, a polyester generally comprises, at the end of preparation, a reactive —CO₂H end group and an —OH end group. It should be noted that this —OH end group will preferably be blocked with an inert group not containing a labile hydrogen, so as not to interfere with the reaction for grafting onto the starting cellulose or the starting cellulose derivative (cellulose ester or ether).

The same comments are applicable for a polyamide, which contains a reactive —CO₂H end group and an —NH₂ end group to be protected with a group that is inert towards the reaction for grafting with the starting cellulose derivative.

A reactive group X₁ may also be introduced onto the polycondensate via the introduction into the reaction medium, during polycondensation, of a reagent bearing the group X₁, which should be inert with respect to the type of polycondensation chosen or inert under the experimental conditions of the polycondensation, and of only one group capable of participating in the polycondensation. This reagent is thus monofunctional with respect to the polycondensation and thus acts as a chain limiter.

This reagent that is monofunctional with respect to the polycondensation and that bears a group that is reactive towards the hydroxyl functions of the starting cellulose derivative is preferably introduced during the polycondensation, in order for the chains of the polymer to end with only one reactive group X₁.

According to a second embodiment, the grafting reaction may consist, in a first stage, in converting all or some of the hydroxyl functions of the starting cellulose or starting cellulose derivative (cellulose ester or ether) into reactive functions, and then, in a second stage, in reacting the said reactive functions with the suitable reactive end groups of polymers comprising the said hydrocarbon-based chain R.

An example that may be mentioned is the following reaction:

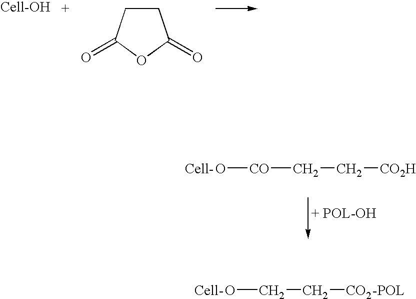

The reaction Cell-OCOCH₂CH₂—CO₂H with Cell-OH may take place, but conditions are used under which this reaction is minimized (crosslinking is avoided).

According to the invention, the polymer grafts may be:

polyolefins (homopolymers or copolymers), which are preferably semi-crystalline;

polydienes, which are preferably hydrogenated;

lipophilic polycondensates such as:

polyesters, polyamides, polyurethanes, polyureas, copolymers (urea/urethane) or polyethers, which are all lipophilic.

II) Liquid Fatty Phase

The liquid fatty phase of the composition according to the invention comprises at least one fatty substance that is liquid at room temperature (25° C.) and atmospheric pressure (10⁵ Pa), also known as an oil.

The liquid fatty phase of the composition may be a continuous fatty phase.

The oils may be volatile or non-volatile, and polar or apolar.

The composition according to the invention advantageously comprises at least one volatile oil.

For the purposes of the invention, the term “volatile oil” means any non-aqueous medium capable of evaporating on contact with keratin materials in less than one hour, at room temperature and atmospheric pressure. The volatile oil(s) of the invention is (are) volatile cosmetic oils, which are liquid at room temperature, having a non-zero vapour pressure, at room temperature and atmospheric pressure, ranging from 0.13 Pa to 40 000 Pa (10⁻³ to 300 mmHg), in particular ranging from 1.3 Pa to 13 000 Pa (0.01 to 100 mmHg) and more particularly ranging from 1.3 Pa to 1300 Pa (0.01 to 10 mmHg).

The term “non-volatile oil” means an oil that remains on keratin materials at room temperature and atmospheric pressure for at least several hours, and that especially has a vapour pressure of less than 0.13 Pa (0.01 mmHg).

These oils may be hydrocarbon-based oils or silicone oils, or mixtures thereof.

The term “hydrocarbon-based oil” means an oil mainly containing hydrogen and carbon atoms, and possibly oxygen, nitrogen, sulfur or phosphorus atoms. The volatile hydrocarbon-based oils may be chosen from hydrocarbon-based oils containing from 8 to 16 carbon atoms, and especially branched C₈-C₁₆ alkanes, for instance C₈-C₁₆ isoalkanes of petroleum origin (also known as isoparaffins), for instance isododecane (also known as 2,2,4,4,6-pentamethylheptane), isodecane and isohexadecane, for example the oils sold under the trade names Isopar and Permethyl.

A volatile oil chosen from C₈-C₁₆ branched alkanes, for instance C₈-C₁₆ isoparaffins such as isododecane, isodecane and isohexadecane, is preferably used.

Volatile oils that may also be used include volatile silicones, for instance linear or cyclic volatile silicone oils, especially those with a viscosity ≦5 centistokes (5×10⁻⁶ m²/s) and especially containing from 2 to 10 silicon atoms, these silicones optionally comprising alkyl or alkoxy groups containing from 1 to 10 carbon atoms. As volatile silicone oils that may be used in the invention, mention may be made especially of octamethylcyclotetrasiloxane, decamethylcyclopentasiloxane, dodecamethyl-cyclohexasiloxane, heptamethylhexyltrisiloxane, hepta-methyloctyltrisiloxane, hexamethyldisiloxane, octamethyl-trisiloxane, decamethyltetrasiloxane and dodeca-methylpentasiloxane, and mixtures thereof.

The volatile oil may be present in the composition according to the invention in a content ranging from 0.1% to 95% by weight, preferably from 1% to 65% by weight and better still from 2% to 50% by weight relative to the weight of the composition.

The composition may also comprise at least one non-volatile oil, chosen especially from non-volatile hydrocarbon-based oils and/or silicone oils.

Non-volatile hydrocarbon-based oils that may especially be mentioned include:
hydrocarbon-based oils of animal origin;
hydrocarbon-based oils of plant origin, such as triglycerides consisting of fatty acid esters of glycerol, the fatty acids of which may have varied chain lengths of from C₄ to C₂₄, these chains possibly being linear or branched, and saturated or unsaturated; these oils are especially wheatgerm oil, sunflower oil, grapeseed oil, sesame seed oil, corn oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppyseed oil, pumpkin oil, marrow oil, blackcurrant oil, evening primrose oil, millet oil, barley oil, quinoa oil, rye oil, safflower oil, candlenut oil, passionflower oil or musk rose oil; or alternatively caprylic/capric acid triglycerides, for instance those sold by the company Stearineries Dubois or those sold under the names Miglyol 810, 812 and 818 by the company Dynamit Nobel;
synthetic ethers containing from 10 to 40 carbon atoms;
linear or branched hydrocarbons of mineral or synthetic origin, such as petroleum jelly, polydecenes, hydrogenated polyisobutene such as parleam, squalane and liquid paraffins, and mixtures thereof;
synthetic esters, for instance oils of formula R₁COOR₂ in which R₁ represents a linear or branched fatty acid residue containing from 1 to 40 carbon atoms and R₂ represents a hydrocarbon-based chain, especially a branched chain, containing from 1 to 40 carbon atoms, on condition that R₁+R₂≧10, for instance purcellin oil (cetostearyl octanoate), isopropyl myristate, isopropyl palmitate, C₁₂ to C₁₅ alkyl benzoate, hexyl laurate, diisopropyl adipate, isononyl isononanoate, 2-ethylhexyl palmitate, isostearyl isostearate, and alcohol or polyalcohol octanoates, decanoates or ricinoleates, for instance propylene glycol dioctanoate; hydroxylated esters, for instance isostearyl lactate or diisostearyl malate; and pentaerythritol esters;
fatty alcohols that are liquid at room temperature, with a branched and/or unsaturated carbon-based chain containing from 12 to 26 carbon atoms, for instance octyldodecanol, isostearyl alcohol, oleyl alcohol, 2-hexyldecanol, 2-butyloctanol or 2-undecylpentadecanol;
higher fatty acids such as oleic acid, linoleic acid or linolenic acid;
and mixtures thereof.

The non-volatile silicone oils that may be used in the composition according to the invention may be non-volatile polydimethylsiloxanes (PDMS), polydimethylsiloxanes comprising alkyl or alkoxy groups, which are pendent and/or at the end of a silicone chain, these groups each containing from 2 to 24 carbon atoms, phenyl silicones, for instance phenyl trimethicones, phenyl dimethicones, phenyltrimethyl-siloxydiphenylsiloxanes, diphenyl dimethicones, diphenylmethyldiphenyltrisiloxanes and 2-phenylethyl trimethylsiloxysilicates, and mixtures thereof.

The non-volatile oils may be present in the composition according to the invention in a content ranging from 0.01% to 95% by weight, preferably from 0.1% to 80% by weight and better still from 1% to 50% by weight (especially 0.1% to 10%) relative to the total weight of the composition.

The liquid fatty phase may represent from 0.01% to 98% by weight, preferably from 0.05% to 75% and better still from 1% to 60% by weight relative to the total weight of the composition.

The composition according to the invention may comprise an aqueous phase consisting essentially of water or of a mixture of water and of water-miscible solvent (miscibility in water of greater than 50% by weight at 25° C.), for instance lower monoalcohols containing from 1 to 5 carbon atoms such as ethanol or isopropanol, glycols containing from 2 to 8 carbon atoms such as propylene glycol, ethylene glycol, 1,3-butylene glycol or dipropylene glycol, C₃-C₄ ketones and C₂-C₄ aldehydes, and mixtures thereof.

The aqueous phase (water and optionally the water-miscible solvent) may be present in a content ranging from 0.1% to 65% by weight, preferably ranging from 1% to 55% by weight and better still from 5% to 50% by weight relative to the total weight of the composition.

The water and/or the water-soluble solvent(s) may be introduced in unmodified form into the formulation according to the invention or may be incorporated by means of one or more constituent ingredients of the said composition. Thus, water may especially be introduced into the composition by means of introducing a latex or pseudolatex, i.e. an aqueous dispersion of polymer particles.

According to one embodiment, the composition according to the invention is anhydrous. The term “anhydrous composition” means a composition comprising an aqueous phase as defined above in a proportion of less than or equal to 10%, preferably less than or equal to 5% and better still less than or equal to 3%, or even free of water.

The composition according to the invention comprises a cosmetically acceptable medium, i.e. a medium that is compatible with keratin materials (acceptable tolerance, toxicology and feel).

The composition according to the invention may also comprise at least one fatty substance that is solid at room temperature, chosen especially from waxes, pasty fatty substances and gums, and mixtures thereof. These fatty substances may be of animal, plant, mineral or synthetic origin.

Waxes

The composition according to the invention may comprise a wax or a mixture of waxes.

The wax under consideration in the context of the present invention is in general a lipophilic compound, which is solid at room temperature (25° C.), with a reversible solid/liquid change of state, having a melting point of greater than or equal to 30° C. that may be up to 200° C.

By bringing the wax to the liquid state (melting), it is possible to make it miscible with the oils and to form a microscopically homogeneous mixture, but on returning the temperature of the mixture to room temperature, recrystallization of the wax in the oils of the mixture is obtained.

In particular, the waxes that are suitable for the invention may have a melting point of greater than about 30° C., preferably greater than 45° C. and in particular greater than 55° C.

The melting point of the wax may be measured using a differential scanning calorimeter (DSC), for example the calorimeter sold under the name DSC 30 by the company Mettler.

The measuring protocol is as follows:

A 15 mg sample of product placed in a crucible is subjected to a first temperature rise ranging from 0° C. to 120° C., at a heating rate of 10° C./minute, it is then cooled from 120° C. to 0° C. at a cooling rate of 10° C./minute and is finally subjected to a second temperature rise ranging from 0° C. to 120° C. at a heating rate of 5° C./minute. During the second temperature rise, the variation of the difference in power absorbed by the empty crucible and by the crucible containing the sample of product is measured as a function of the temperature. The melting point of the compound is the temperature value corresponding to the top of the peak of the curve representing the variation in the difference in power absorbed as a function of the temperature.

The waxes that may be used in the compositions according to the invention are chosen from waxes that are solid and rigid at room temperature, of animal, plant, mineral or synthetic origin, and mixtures thereof.

The wax may also have a hardness ranging from 0.05 MPa to 30 MPa and preferably ranging from 6 MPa to 15 MPa. The hardness is determined by measuring the compression force, measured at 20° C. using the texturometer sold under the name TA-TX2i by the company Rheo, equipped with a stainless-steel cylinder 2 mm in diameter travelling at a measuring speed of 0.1 mm/s, and penetrating the wax to a penetration depth of 0.3 mm.

The measuring protocol is as follows:

The wax is melted at a temperature equal to the melting point of the wax +20° C. The molten wax is poured into a container 30 mm in diameter and 20 mm deep. The wax is recrystallized at room temperature (25° C.) for 24 hours and is then kept at 20° C. for at least 1 hour before performing the hardness measurement. The hardness value is the maximum compression force measured divided by the surface area of the texturometer cylinder in contact with the wax.

Hydrocarbon-based waxes, for instance beeswax or lanolin wax; rice wax, Japan wax, carnauba wax, candelilla wax, microcrystalline waxes, paraffins and ozokerite; polyethylene waxes, the waxes obtained by Fisher-Tropsch synthesis and waxy copolymers, and also esters thereof, may especially be used.

Mention may also be made of waxes obtained by catalytic hydrogenation of animal or plant oils containing linear or branched C₈-C₃₂ fatty chains.

Among these waxes that may especially be mentioned are hydrogenated jojoba oil, isomerized jojoba oil such as the trans-isomerized partially hydrogenated jojoba oil manufactured or sold by the company Desert Whale under the commercial reference Iso-Jojoba-50®, hydrogenated sunflower oil, hydrogenated castor oil, hydrogenated coconut oil, hydrogenated lanolin oil and bis(1,1,1-trimethylolpropane) tetrastearate sold under the name “Hest 2T-4S” by the company Heterene, bis(1,1,1-trimethylolpropane) tetrabehenate sold under the name Hest 2T-4B by the company Heterene.

Mention may also be made of silicone waxes, for instance alkyl or alkoxy dimethicones containing from 16 to 45 carbon atoms, and fluoro waxes.

The wax obtained by hydrogenation of olive oil esterified with stearyl alcohol, sold under the name “Phytowax Olive 18 L57” or else the waxes obtained by hydrogenation of castor oil esterified with cetyl alcohol sold under the names “Phytowax ricin 16L64 and 22L73” by the company Sophim may also be used. Such waxes are described in patent application FR-A-2 792 190.

According to one particular embodiment, the compositions according to the invention may comprise at least one “tacky” wax, i.e. a wax with a tack of greater than or equal to 0.7 N·s and a hardness of less than or equal to 3.5 MPa.

The tack of the wax is determined by measuring the change in force (compression force or stretching force) as a function of time, at 20° C., using the texturometer sold under the name “TA-TX2i®” by the company Rheo, equipped with a conical acrylic polymer spindle forming an angle of 45°.

The measuring protocol is as follows:

The wax is melted at a temperature equal to the melting point of the wax +10° C. The molten wax is poured into a container 25 mm in diameter and 20 mm deep. The wax is recrystallized at room temperature (25° C.) for 24 hours such that the surface of the wax is flat and smooth, and the wax is then stored for at least 1 hour at 20° C. before measuring the tack.

The texturometer spindle is displaced at a speed of 0.5 mm/s then penetrates the wax to a penetration depth of 2 mm. When the spindle has penetrated the wax to a depth of 2 mm, the spindle is held still for 1 second (corresponding to the relaxation time) and is then withdrawn at a speed of 0.5 mm/s.

During the relaxation time, the force (compression force) decreases greatly until it becomes zero, and then, during the withdrawal of the spindle, the force (stretching force) becomes negative and then rises again to the value 0. The tack corresponds to the integral of the curve of the force as a function of time for the part of the curve corresponding to negative values of the force (stretching force). The tack value is expressed in N·s.

The hardness is measured according to the protocol described previously.

Tacky waxes that may be used include a C₂₀-C₄₀ alkyl (hydroxystearyloxy)stearate (the alkyl group containing from 20 to 40 carbon atoms), for instance the waxes sold under the names “Kester Wax K 82 P®” and “Kester Wax K 80 P®” by the company Koster Keunen.

The microcrystalline wax sold under the reference SP18 by the company Strahl & Pitsch, which has a hardness of about 0.46 MPa and a tack value of about 1 N·s, may also be mentioned.

The waxes mentioned above generally have a starting melting point of less than 45° C.

The wax(es) may be in the form of an aqueous microdispersion of wax. The expression “aqueous microdispersion of wax” means an aqueous dispersion of wax particles in which the size of the said wax particles is less than or equal to about 1 μm.

Wax microdispersions are stable dispersions of colloidal wax particles, and are described especially in “Microemulsions Theory and Practice”, L. M. Prince Ed., Academic Press (1977) pages 21-32.

In particular, these wax microdispersions may be obtained by melting the wax in the presence of a surfactant, and optionally of a portion of water, followed by gradual addition of hot water with stirring. The intermediate formation of an emulsion of the water-in-oil type is observed, followed by a phase inversion, with final production of a microemulsion of the oil-in-water type. On cooling, a stable microdispersion of solid wax colloidal particles is obtained.

The wax microdispersions may also be obtained by stirring the mixture of wax, surfactant and water using stirring means such as ultrasound, high-pressure homogenizers or turbomixers.

The particles of the wax microdispersion preferably have mean sizes of less than 1 μm (especially ranging from 0.02 μm to 0.99 μm) and preferably less than 0.5 μm (especially ranging from 0.06 μm to 0.5 μm).

These particles consist essentially of a wax or a mixture of waxes. However, they may comprise a small proportion of oily and/or pasty fatty additives, a surfactant and/or a common liposoluble additive/active agent.

The term “pasty fatty substance” means a lipophilic fatty compound comprising at a temperature of 23° C. a liquid fraction and a solid fraction.

The said pasty compound preferably has a hardness at 20° C. ranging from 0.001 to 0.5 MPa and preferably from 0.002 to 0.4 MPa.

The hardness is measured according to a method of penetration of a probe in a sample of compound and in particular using a texture analyser (for example the TA-XT2i machine from Rheo) equipped with a stainless-steel spindle 2 mm in diameter. The hardness measurement is performed at 20° C. at the centre of five samples. The spindle is introduced into each sample at a pre-speed of 1 mm/s and then at a measuring speed of 0.1 mm/s, the penetration depth being 0.3 mm. The hardness value revealed is that of the maximum peak.

The liquid fraction of the pasty compound measured at 23° C. preferably represents 9% to 97% by weight of the compound. This liquid fraction at 23° C. preferably represents between 15% and 85% and more preferably between 40% and 85% by weight. The liquid fraction by weight of the pasty compound at 23° C. is equal to the ratio of the heat of fusion consumed at 23° C. to the heat of fusion of the pasty compound.

The heat of fusion of the pasty compound is the heat consumed by the compound to change from the solid state to the liquid state. The pasty compound is said to be in the solid state when all of its mass is in solid crystalline form. The pasty compound is said to be in the liquid state when all of its mass is in liquid form.

The heat of fusion of the pasty compound is equal to the area under the curve of the thermogram obtained using a differential scanning calorimeter (DSC), such as the calorimeter sold under the name MDSC 2920 by the company TA Instrument, with a temperature rise of 5 or 10° C. per minute, according to standard ISO 11357-3:1999. The heat of fusion of the pasty compound is the amount of energy required to make the compound change from the solid state to the liquid state. It is expressed in J/g.

The heat of fusion consumed at 23° C. is the amount of energy absorbed by the sample to change from the solid state to the state that it has at 23° C., consisting of a liquid fraction and a solid fraction.

The liquid fraction of the pasty compound, measured at 32° C., preferably represents from 30% to 100% by weight of the compound, preferably from 80% to 100% and more preferably from 90% to 100% by weight of the compound. When the liquid fraction of the pasty compound measured at 32° C. is equal to 100%, the temperature of the end of the melting range of the pasty compound is less than or equal to 32° C.

The liquid fraction of the pasty compound measured at 32° C. is equal to the ratio of the heat of fusion consumed at 32° C. to the heat of fusion of the pasty compound. The heat of fusion consumed at 32° C. is calculated in the same manner as the heat of fusion consumed at 23° C.

The pasty substances are generally hydrocarbon-based compounds, for instance lanolins and derivatives thereof, or alternatively PDMSs.

The nature and amount of the solid substances depend on the desired mechanical properties and textures.

As a guide, the composition may contain from 0.1% to 50% by weight, better still from 1% to 40% and even better still from 5% to 20% by weight of waxes, relative to the total weight of the composition.

The composition according to the invention may also comprise, besides the modified cellulose or the modified cellulose derivative, an “additional” film-forming polymer.

Among the additional film-forming polymers that may be used in the composition of the present invention, mention may be made of synthetic polymers, of free-radical type or of polycondensate type, and polymers of natural origin, and mixtures thereof. Film-forming polymers that may be mentioned in particular include acrylic polymers, polyurethanes, polyesters, polyamides, polyureas and cellulose-based polymers other than liposoluble modified cellulose derivatives.

The additional film-forming polymers may be soluble or dispersible in a liquid fatty phase, which may be the liquid fatty phase of the composition. They may also be chosen from film-forming polymers that are water-soluble or dispersible in an aqueous phase (also known as latices).

The additional film-forming polymer may be present in a content ranging from 0.1% to 30% by weight and better still from 0.5% to 15% by weight of solids, relative to the total weight of the composition.

The composition according to the invention may also comprise one or more dyestuffs chosen from water-soluble dyes, and pulverulent dyestuffs, for instance pigments, nacres and flakes that are well known to those skilled in the art. The dyestuffs may be present in the composition in a content ranging from 0.01% to 50% by weight, preferably from 0.01% to 30% by weight and better still from 1% to 25% by weight relative to the weight of the composition.

The term “pigments” should be understood as meaning white or coloured, mineral or organic particles of any form, which are insoluble in the physiological medium, and which are intended to colour the composition.

The term “nacres” should be understood as meaning iridescent particles of any form, especially produced by certain molluscs in their shell, or alternatively synthesized.

The pigments may be white or coloured, and mineral and/or organic. Among the mineral pigments that may be mentioned are titanium dioxide, optionally surface-treated, zirconium oxide or cerium oxide, and also zinc oxide, iron oxide (black, yellow or red) or chromium oxide, manganese violet, ultramarine blue, chromium hydrate and ferric blue, and metal powders, for instance aluminium powder or copper powder.

Among the organic pigments that may be mentioned are carbon black, pigments of D & C type, and lakes based on cochineal carmine or on barium, strontium, calcium or aluminium.

Mention may also be made of pigments with an effect, such as particles comprising a natural or synthetic, organic or mineral substrate, for instance glass, acrylic resins, polyester, polyurethane, polyethylene terephthalate, ceramics or aluminas, the said substrate possibly being coated with metallic substances, for instance aluminium, gold, silver, platinum, copper, bronze or metal oxides, for instance titanium dioxide, iron oxide or chromium oxide, and mixtures thereof.

The nacreous pigments may be chosen from white nacreous pigments such as mica coated with titanium or with bismuth oxychloride, coloured nacreous pigments such as titanium mica coated with iron oxides, titanium mica coated especially with ferric blue or with chromium oxide, titanium mica coated with an organic pigment of the abovementioned type and also nacreous pigments based on bismuth oxychloride. It is also possible to use interference pigments, especially liquid crystal pigments or multilayer pigments.

The liposoluble dyestuffs are, for example, Sudan Red, D & C Red 17, D & C Green 6, β-carotene, soybean oil, Sudan Brown, D & C Yellow 11, D & C Violet 2, D & C Orange 5, quinoline yellow or annatto. The water-soluble dyes are, for example, beetroot juice, methylene blue, the disodium salt of ponceau, the disodium salt of alizarin green, quinoline yellow, the trisodium salt of amaranth, the disodium salt of tartrazine, the monosodium salt of rhodamine, the disodium salt of fuchsin, or xanthophyll.

The composition according to the invention may comprise at least one filler, especially in a content ranging from 0.01% to 50% by weight and preferably ranging from 0.01% to 30% by weight relative to the total weight of the composition. The term “fillers” should be understood as meaning colourless or white, mineral or synthetic particles of any form, which are insoluble in the medium of the composition irrespective of the temperature at which the composition is manufactured. These fillers serve especially to modify the rheology or texture of the composition.

The fillers may be mineral or organic and of any form, platelet-shaped, spherical or oblong, irrespective of the crystallographic form (for example lamellar, cubic, hexagonal, orthorhombic, etc.). Mention may be made of talc, mica, silica, kaolin, polyamide powder, for instance Nylon® (Orgasol® from Atochem), poly-β-alanine powder and polyethylene powder, tetrafluoroethylene polymer powders, (Teflon®) lauroyllysine, starch, boron nitride, expanded hollow polymer microspheres such as those made of polyvinylidene chloride/acrylonitrile, for instance Expancel® (Nobel Industrie), acrylic acid copolymers (Polytrap® from Dow Corning) and silicone resin microbeads (for example Tospearls® from Toshiba), elastomeric polyorganosiloxane particles, precipitated calcium carbonate, magnesium carbonate, magnesium hydrocarbonate, hydroxyapatite, hollow silica microspheres (Silica Beads® from Maprecos), glass or ceramic microcapsules, and metal soaps derived from organic carboxylic acids containing from 8 to 22 carbon atoms and preferably from 12 to 18 carbon atoms, for example zinc, magnesium or lithium stearate, zinc laurate or magnesium myristate.

The composition according to the invention may also contain ingredients commonly used in cosmetics, such as vitamins, thickeners, gelling agents, trace elements, softeners, sequestering agents, fragrances, acidifying or basifying agents, preserving agents, sunscreens, surfactants, antioxidants, fibres, agents for preventing hair loss, eyelash care agents, antidandruff agents and propellants, or mixtures thereof.

The term “fibre” should be understood as meaning an object of length L and diameter D such that L is very much greater than D, D being the diameter of the circle within which the cross section of the fibre is inscribed. In particular, the L/D ratio (or form factor) is chosen within the range from 3.5 to 2500, preferably from 5 to 500 and better still from 5 to 150.

In particular, the fibres have a length ranging from 1 um to 10 mm, preferably from 0.1 mm to 5 mm and better still from 0.3 mm to 3 mm.

The fibres that may be used in the composition of the invention may be chosen from rigid and non-rigid fibres, and they may be of synthetic or natural, mineral or organic origin.

The fibres may be present in the composition according to the invention in a content ranging from 0.1% to 10% by weight and better still from 0.5% to 5% by weight relative to the total weight of the composition.

The gelling agents that may be used in the compositions according to the invention may be organic or mineral, and polymeric or molecular, hydrophilic or lipophilic gelling agents.

Mineral lipophilic gelling agents that may be mentioned include optionally modified clays, for instance hectorites modified with a C₁₀ to C₂₂ fatty acid ammonium chloride, for instance hectorite modified with distearyldimethylammonium chloride, for instance the product sold under the name “Bentone 38V®” by the company Elementis.

Mention may also be made of fumed silica optionally subjected to a hydrophobic surface treatment, the particle size of which is less than 1 μm. Specifically, it is possible to chemically modify the surface of the silica, by chemical reaction generating a reduction in the number of silanol groups present at the surface of the silica. It is especially possible to substitute silanol groups with hydrophobic groups: a hydrophobic silica is then obtained. The hydrophobic groups may be:

trimethylsiloxyl groups, which are obtained especially by treating fumed silica in the presence of hexamethyldisilazane. Silicas thus treated are known as “silica silylate” according to the CTFA (6th edition, 1995). They are sold, for example, under the references “Aerosil R812®” by the company Degussa, and “Cab-O-Sil TS-530®” by the company Cabot;

dimethylsilyloxyl or polydimethylsiloxane groups, which are obtained especially by treating fumed silica in the presence of polydimethylsiloxane or dimethyldichlorosilane. Silicas thus treated are known as “silica dimethyl silylate” according to the CTFA (6th edition, 1995). They are sold, for example, under the references “Aerosil R972®” and “Aerosil R974®” by the company Degussa, and “Cab-O-Sil TS-610®” and “Cab-O-Sil TS-720®” by the company Cabot.

The hydrophobic fumed silica particularly has a particle size that may be nanometric to micrometric, for example ranging from about 5 to 200 nm.

The polymeric organic lipophilic gelling agents are, for example, partially or totally crosslinked elastomeric organopolysiloxanes of three-dimensional structure, for instance those sold under the names KSG6®, KSG16® and KSG18® from Shin-Etsu, Trefil E-505C® or Trefil E-506C ® from Dow Corning, Gransil SR—CYC®, SR DMF 10®, SR-DC556®, SR 5CYC gel®, SR DMF 10 gel® and SR DC 556 gel® from Grant Industries and SF 1204® and JK 113® from General Electric; block copolymers of “diblock” or “triblock” type, of the polystyrene/polyisoprene or polystyrene/polybutadiene type, such as the products sold under the name Luvitol HSB® by the company BASF, of the polystyrene/copoly(ethylene-propylene) type, such as the products sold under the name Kraton® by the company Shell Chemical Co., or of the polystyrene/copoly(ethylene-butylene) type; polycondensates of polyamide type resulting from condensation between (α) at least one acid chosen from dicarboxylic acids containing at least 32 carbon atoms, such as fatty acid dimers, and (β) an alkylenediamine and in particular ethylenediamine, in which the polyamide polymer comprises at least one carboxylic acid end group esterified or amidated with at least one saturated and linear monoalcohol or one saturated and linear monoamine containing from 12 to 30 carbon atoms, and in particular ethylenediamine/stearyl dilinoleate copolymers such as the product sold under the name Uniclear 100 VG® by the company Arizona Chemical; ethylcellulose, for instance the product sold under the name Ethocel by Dow Chemical; galactomannans comprising from one to six and in particular from two to four hydroxyl groups per saccharide, substituted with a saturated or unsaturated alkyl chain, for instance guar gum alkylated with C₁ to C₆, and in particular C₁ to C₃, alkyl chains, and mixtures thereof.

Among the gelling agents that may be used in the compositions according to the invention, mention may also be made of fatty acid esters of dextrin, such as dextrin palmitates, especially the products sold under the name Rheopearl TL® or Rheopearl KL® by the company Chiba Flour.

Mention may also be made of silicone polyamides of the polyorganosiloxane type. These silicone polymers may belong to the following two families:
1) polyorganosiloxanes comprising at least two groups capable of establishing hydrogen interactions, these two groups being located in the polymer chain, and/or
2) polyorganosiloxanes comprising at least two groups capable of establishing hydrogen interactions, these two groups being located on grafts or branches.

These polymers are described in documents U.S. Pat. No. 5,874,069, U.S. Pat. No. 5,919,441, U.S. Pat. No. 6,051,216 and U.S. Pat. No. 5,981,680.

When the composition according to the invention comprises an aqueous medium, it may comprise a hydrophilic or water-soluble gelling agent.

Hydrophilic or water-soluble gelling agents that may be mentioned include:

homopolymers or copolymers of acrylic or methacrylic acid or the salts and esters thereof, and in particular the products sold under the names “Versicol F” or “Versicol K” by the company Allied Colloid, “Ultrahold 8” by the company Ciba-Geigy, and the polyacrylic acids of Synthalen K type;

copolymers of acrylic acid and of acrylamide sold in the form of the sodium salt thereof under the names “Reten” by the company Hercules, sodium polymethacrylate sold under the name “Darvan No. 7” by the company Vanderbilt, and the sodium salts of polyhydroxycarboxylic acids sold under the name “Hydagen F” by the company Henkel;

polyacrylic acid/alkyl acrylate copolymers of the Pemulen type;

AMPS (polyacrylamidomethylpropanesulfonic acid partially neutralized with ammonia and highly crosslinked) sold by the company Clariant;

AMPS/acrylamide copolymers of the Sepigel or Simulgel type, sold by the company SEPPIC, and

AMPS/polyoxyethylenated alkyl methacrylate copolymers (crosslinked or non-crosslinked); and mixtures thereof.

As other examples of water-soluble gelling polymers, mention may be made of:

proteins, for instance proteins of plant origin, such as wheat or soybean proteins; proteins of animal origin such as keratins, for example keratin hydrolysates and sulfonic keratins;

anionic, cationic, amphoteric or nonionic chitin or chitosan polymers;

non-liposoluble cellulose polymers such as hydroxyethylcellulose, hydroxypropylcellulose, methylcellulose, ethylhydroxyethylcellulose and carboxymethylcellulose, and also quaternized cellulose derivatives;

vinyl polymers, for instance polyvinylpyrrolidones, copolymers of methyl vinyl ether and of malic anhydride, the copolymer of vinyl acetate and of crotonic acid, copolymers of vinylpyrrolidone and of vinyl acetate; copolymers of vinylpyrrolidone and of caprolactam; polyvinyl alcohol;

associative polyurethanes such as the C₁₆—OE₁₂₀-C₁₆ polymer from the company Servo Delden (sold under the name Ser Ad FX1100, which is a molecule containing urethane functions and having a weight-average molecular weight of 1300), OE being an oxyethylene unit, Rheolate 205 containing urea functions, sold by the company Rheox, or Rheolate 208 or 204 (these polymers being sold in pure form) or DW 1206B from Röhm & Haas, containing a C₂₀ alkyl chain and a urethane bond, sold at a solids content of 20% in water. It is also possible to use solutions or dispersions of these associative polyurethanes, especially in water or in aqueous-alcoholic medium. Examples of such polymers that may be mentioned include Ser Ad FX1010, Ser Ad FX1035 and Ser Ad 1070 from the company Servo Delden, and Rheolate 255, Rheolate 278 and Rheolate 244 sold by the company Rheox. It is also possible to use the product DW 1206F and DW 1206J, and also Acrysol RM 184 or Acrysol 44 from the company Röhm & Haas, or Borchigel LW 44 from the company Borchers;

optionally modified polymers of natural origin, such as:
gum arabics, guar gum, xanthan derivatives and karaya gum;
alginates and carrageenans;
glycoaminoglycans, and hyaluronic acid and its derivatives;
sandarac gum, dammar resins, elemi gums and copal resins;
deoxyribonucleic acid;
mucopolysaccharides such as hyaluronic acid and chondroitin sulfates, and mixtures thereof.

The lipophilic or hydrophilic gelling agents may be present in the composition according to the invention in a content ranging from 0.05% to 40% by weight, preferably from 0.5% to 20% and better still from 1% to 15% by weight relative to the total weight of the composition.

The composition according to the invention may contain emulsifying surfactants, which are especially present in a proportion ranging from 0.5% to 30% by weight, better still from 1% to 15% and even better still from 3% to 10% relative to the total weight of the composition. These surfactants may be chosen from anionic, cationic and nonionic surfactants. Reference may be made to the document “Encyclopedia of Chemical Technology, Kirk-Othmer”, Volume 22, pp. 333-432, 3rd Edition, 1979, Wiley, for the definition of the properties and functions (emulsifying) of surfactants, in particular pp. 347-377 of this reference, for the anionic and nonionic surfactants.

As surfactants that may be used in the composition according to the invention, mention may be made of:
a) nonionic surfactants with an HLB of greater than or equal to 8 at 25° C., used alone or as a mixture; mention may be made especially of:
   oxyethylenated and/or oxypropylenated ethers (which may comprise from 1 to 150 oxyethylene and/or oxypropylene groups) of glycerol;
   oxyethylenated and/or oxypropylenated ethers (which may comprise from 1 to 150 oxyethylene and/or oxypropylene groups) of fatty alcohols (especially of C₈-C₂₄ and preferably C₁₂-C₁₈ alcohol), such as oxyethylenated cetearyl alcohol ether containing 30 oxyethylene groups (CTFA name “Ceteareth-30”) and the oxyethylenated ether of the mixture of C₁₂-C₁₅ fatty alcohols comprising 7 oxyethylene groups (CTFA name “C12-15 Pareth-7” sold under the name “Neodol 25-7” by Shell Chemicals);
   fatty acid esters (especially of a C₈-C₂₄ and preferably C₁₆-C₂₂ acid) of polyethylene glycol (which may comprise from 1 to 150 ethylene glycol units), such as PEG-50 stearate and PEG-40 monostearate sold under the name Myrj 52P by the company ICI Uniqema;
   fatty acid esters (especially of a C₈-C₂₄ and preferably C₁₆-C₂₂ acid) of oxyethylenated and/or oxypropylenated glyceryl ethers (which may comprise from 1 to 150 oxyethylene and/or oxypropylene groups), for instance PEG-200 glyceryl monostearate sold under the name “Simulsol 220™” by the company SEPPIC; glyceryl stearate polyethoxylated with 30 ethylene oxide groups, for instance the product Tagat S sold by the company Goldschmidt, glyceryl oleate polyethoxylated with 30 ethylene oxide groups, for instance the product Tagat O sold by the company Goldschmidt, glyceryl cocoate polyethoxylated with 30 ethylene oxide groups, for instance the product Varionic LI 13 sold by the company Sherex, glyceryl isostearate polyethoxylated with 30 ethylene oxide groups, for instance the product Tagat L sold by the company Goldschmidt, and glyceryl laurate polyethoxylated with 30 ethylene oxide groups, for instance the product Tagat I from the company Goldschmidt;
   fatty acid esters (especially of a C₈-C₂₄ and preferably C₁₆-C₂₂ acid) of oxyethylenated and/or oxypropylenated sorbitol ethers (which may comprise from 1 to 150 oxyethylene and/or oxypropylene groups), for instance polysorbate 60 sold under the name “Tween 60” by the company Uniqema;
   dimethicone copolyol, such as the product sold under the name “Q2-5220” by the company Dow Corning;
   dimethicone copolyol benzoate (Finsolv SLB 101 and 201 by the company Finetex);
   copolymers of propylene oxide and of ethylene oxide, also known as EO/PO polycondensates, for instance the polyethylene glycol/polypropylene glycol/polyethylene glycol triblock polycondensates sold under the names “Synperonic”, for instance “Synperonic PE/L44” and “Synperonic PE/F127”, by the company ICI, and mixtures thereof;
   and mixtures thereof.
b) nonionic surfactants with an HLB of less than 8 at 25° C., optionally combined with one or more nonionic surfactants with an HLB of greater than 8 at 25° C., as mentioned above, such as:
   saccharide esters and ethers, such as sucrose stearate, sucrose cocoate and sorbitan stearate, and mixtures thereof, for instance Arlatone 2121 sold by the company ICI;
   fatty acid esters (especially of a C₈-C₂₄ and preferably C₁₆-C₂₂ acid) of polyols, especially of glycerol or of sorbitol, such as glyceryl stearate, glyceryl stearate such as the product sold under the name Tegin M by the company Goldschmidt, glyceryl laurate such as the product sold under the name Imwitor 312 by the company Hüls, polyglyceryl-2 stearate, sorbitan tristearate or glyceryl ricinoleate;
   the mixture of cyclomethicone/dimethicone copolyol sold under the name “Q2-3225C” by the company Dow Corning.
c) anionic surfactants such as:
   C₁₆-C₃₀ fatty acid salts, especially those derived from amines, for instance triethanolamine stearate;
   polyoxyethylenated fatty acid salts, especially those derived from amines or alkali metal salts, and mixtures thereof;
   phosphoric esters and salts thereof, such as “DEA oleth-10 phosphate” (Crodafos N 10N from the company Croda);
   sulfosuccinates such as “Disodium PEG-5 citrate lauryl sulfosuccinate” and “Disodium ricinoleamido MEA sulfosuccinate”;
   alkyl ether sulfates, such as sodium lauryl ether sulfate;
   isethionates;
   acylglutamates such as “Disodium hydrogenated tallow glutamate” (Amisoft HS-21 R sold by the company Ajinomoto), and mixtures thereof.

Triethanolamine stearate is most particularly suitable for the invention. This is generally obtained by simple mixing of stearic acid and triethanolamine.

Surfactants that allow an oil-in-water or wax-in-water emulsion to be obtained are preferably used.

The composition according to the invention may especially be in the form of a suspension, a dispersion, a solution, a gel, an emulsion, especially an oil-in-water (O/W) emulsion, a water-in-oil (W/O) or a multiple emulsion (W/O/W, polyol/O/W or O/W/O), or in the form of a cream, a paste, a mousse, a dispersion of vesicles, especially of ionic or nonionic lipids, a two-phase or multi-phase lotion, a spray, a powder or a paste, especially a soft paste. The composition may be anhydrous: for example, it may be an anhydrous paste or stick. The composition is preferably a leave-in composition.

A person skilled in the art may choose the appropriate galenical form, and also the method for preparing it, on the basis of his general knowledge, taking into account firstly the nature of the constituents used, especially their solubility in the support, and secondly the intended use of the composition.

The composition according to the invention may especially be in the form of a stick, a suspension, a dispersion, a solution, a gel, an emulsion, especially an oil-in-water (O/W) or water-in-oil (W/O) emulsion, or a multiple emulsion (W/O/W or polyol/O/W or O/W/O), or in the form of a cream, a paste, a mousse, a dispersion of vesicles, especially of ionic or nonionic lipids, a two-phase or multi-phase lotion, a spray, a powder, a paste, especially a soft paste (especially a paste with a dynamic viscosity at 25° C. of about from 0.1 to 40 Pa·s at a shear rate of 200 s⁻¹, after 10 minutes of measurement in cone/plane geometry). The composition may be anhydrous, for example it may be an anhydrous paste.

The composition according to the invention may be a makeup composition, for instance complexion products (foundations), makeup rouges, eyeshadows, lipsticks, concealer products, blushers, mascaras, eyeliners, eyebrow makeup products, lip pencils, eye pencils, nail products, such as nail varnishes, body makeup products or hair makeup products (hair mascara or hair lacquer).

The composition according to the invention may also be a body and facial skincare product, especially an antisun product or a skin colouring product (such as a self-tanning product).

According to one embodiment, one subject of the invention is a composition for coating keratin fibres (such as the eyelashes, the eyebrows or the hair).

Such a composition may be in various forms: for example, in the form of wax-in-water or water-in-wax two-phase emulsions, or aqueous or anhydrous dispersions.

In particular, the composition may be a makeup product for keratin fibres such as the eyelashes (mascara) or a lip or skin makeup product.

According to one variant, a subject of the invention is a cosmetic composition comprising, in a cosmetically acceptable medium, a liquid fatty phase and a liposoluble modified cellulose or cellulose ester, the said modified cellulose or the said modified cellulose ester comprising free hydroxyl functions totally or partially replaced with hydrophobic groups chosen from the radicals of formula —O—Y—R, in which:
R represents a group chosen from:
A) hydrocarbon-based groups containing linear or branched, saturated or unsaturated chains, or saturated or unsaturated cyclic hydrocarbon-based groups, containing from 8 to 50 carbon atoms for the modified cellulose or from 4 to 50 carbon atoms for the modified cellulose ester;

the said groups possibly comprising in their chains one or more aromatic groups and/or one or more hetero atoms chosen from O, N, P, Si and S; the said groups possibly being fluorinated or perfluorinated;
B) groups of polymeric nature chosen from polyolefins, hydrogenated or non-hydrogenated polydienes and lipophilic polycondensates, and mixtures thereof;
Y represents a single bond or a divalent bonding group.

According to another variant, a subject of the invention is an anhydrous cosmetic composition comprising, in a cosmetically acceptable medium, a liquid fatty phase and at least 4% of a liposoluble modified cellulose derivative, the said modified cellulose derivative comprising free hydroxyl functions totally or partially replaced with hydrophobic groups chosen from the radicals of formula —O—Y—R, in which:
R represents a group chosen from:
A) hydrocarbon-based groups containing linear or branched, saturated or unsaturated chains, or saturated or unsaturated cyclic hydrocarbon-based groups, containing from 4 to 50 carbon atoms for the modified cellulose derivative;

the said groups possibly comprising in their chains one or more aromatic groups and/or one or more hetero atoms chosen from O, N, P, Si and S; the said groups possibly being fluorinated or perfluorinated;
B) groups of polymeric nature chosen from polyolefins, hydrogenated or non-hydrogenated polydienes and lipophilic polycondensates, and mixtures thereof;
Y represents a single bond or a divalent bonding group.

A subject of the present invention is also a cosmetic assembly comprising:
a container delimiting at least one compartment, the said container being closed by means of a closing member; and
a composition as described above, placed inside the said compartment.

The container may be in any adequate form. It may especially be in the form of a bottle, a tube, a jar, a case, a box, a sachet or a carton.

The closing member may be in the form of a removable stopper, a lid, a cap, a tear-off strip or a capsule, especially of the type comprising a body attached to the container and a cover cap articulated on the body. It may also be in the form of a member for selectively closing the container, especially a pump, a valve or a flap valve.

The container may be combined with an applicator, especially in the form of a brush comprising an arrangement of bristles maintained by a twisted wire. Such a twisted brush is described especially in patent U.S. Pat. No. 4,887,622. It may also be in the form of a comb comprising a plurality of application members, obtained especially by moulding. Such combs are described, for example, in patent FR 2 796 529. The applicator may be in the form of a fine brush, as described, for example, in patent FR 2 722 380. The applicator may be in the form of a block of foam or of elastomer, a felt or a spatula. The applicator may be free (tuft or sponge) or securely fastened to a rod borne by the closing member, as described, for example, in U.S. Pat. No. 5,492,426. The applicator may be securely fastened to the container, as described, for example, in patent FR 2 761 959.

The product may be contained directly in the container, or indirectly. For example, the product may be arranged on an impregnated support, especially in the form of a wipe or a pad, and arranged (individually or in plurality) in a box or in a sachet. Such a support incorporating the product is described, for example, in patent application WO 01/03538.

The closing member may be coupled to the container by screwing. Alternatively, the coupling between the closing member and the container is done other than by screwing, especially via a bayonet mechanism, by click-fastening, gripping, welding, bonding or by magnetic attraction. The term “click-fastening” in particular means any system involving the crossing of a bead or cord of material by elastic deformation of a portion, especially of the closing member, followed by return to the elastically unconstrained position of the said portion after the crossing of the bead or cord.

The container may be at least partially made of thermoplastic material. Examples of thermoplastic materials that may be mentioned include polypropylene or polyethylene.

Alternatively, the container is made of non-thermoplastic material, especially glass or metal (or alloy).

The container may have rigid walls or deformable walls, especially in the form of a tube or a tubular bottle.

The container may comprise means for distributing or facilitating the distribution of the composition. By way of example, the container may have deformable walls so as to cause the composition to exit in response to a positive pressure inside the container, this positive pressure being caused by elastic (or non-elastic) squeezing of the walls of the container. Alternatively, especially when the product is in the form of a stick, the product may be driven out by a piston mechanism. Still in the case of a stick, especially of makeup product (lipstick, foundation, etc.), the container may comprise a mechanism, especially a rack mechanism, a threaded-rod mechanism or a helical groove mechanism, and may be capable of moving a stick in the direction of the said aperture. Such a mechanism is described, for example, in patent FR 2 806 273 or in patent FR 2 775 566. Such a mechanism for a liquid product is described in patent FR 2 727 609.

The container may consist of a carton with a base delimiting at least one housing containing the composition, and a lid, especially articulated on the base, and capable of at least partially covering the said base. Such a carton is described, for example, in patent application WO 03/018423 or in patent FR 2 791 042.

The container may be equipped with a drainer arranged in the region of the aperture of the container. Such a drainer makes it possible to wipe the applicator and possibly the rod to which it may be securely fastened. Such a drainer is described, for example, in patent FR 2 792 618.

The composition may be at atmospheric pressure inside the container (at room temperature) or pressurized, especially by means of a propellent gas (aerosol). In the latter case, the container is equipped with a valve (of the type used for aerosols).

The content of the patents or patent applications mentioned above is incorporated by reference in the present patent application.

The examples that follow illustrate the compositions according to the invention in a non-limiting manner.

Unless otherwise indicated, the amounts are expressed in grams.

### EXAMPLE 1

### Preparation of a Cellulose Acetobutyrate Containing Isostearyl Ester Side Groups

The reagents used are as follows:

cellulose acetobutyrate CAB 553-0.4 from Eastman, comprising 4.8% by weight of free hydroxyl functions (non-esterified);

isostearyl chloride (C₁₈H₃₅OCl, M=302.5): amount to esterify ⅔ of the residual —OH of the cellulose acetobutyrate=0.188 mol, i.e. 56.87 g,

triethylamine=20 g (qs neutralization of the HCl released during the reaction),

1000 g solvent mixture consisting of methyl ethyl ketone (500 g)+toluene (500 g).

The procedure is as follows:

1800 g of a solvent mixture consisting of 900 g of methyl ethyl ketone and 900 g of toluene are introduced into a 3 L reactor with a central stirrer, a condenser and a nitrogen inlet.

100 g of cellulose acetobutyrate powder or granulate CAB 553-04 are gradually introduced with stirring and at room temperature. The mixture is then heated with stirring at 50° C. for 1 hour to fully dissolve the cellulose ester. When the dissolution is complete, the mixture is cooled to room temperature with continued stirring, and 20 g of triethylamine are added.

A stream of nitrogen is bubbled into the solution with stirring, and the solution is cooled to +5° C. in an ice bath.

From an addition funnel mounted over the reactor, 56.87 g of isostearyl chloride dissolved in a mixture of 100 g of methyl ethyl ketone and 100 g of toluene are added.

When the temperature of the solution contained in the reactor has fallen to +5° C., the isostearyl chloride solution is added dropwise, while keeping the internal temperature below +10° C. The addition time is 1 hour 30 minutes.

Once the addition is complete, the mixture is allowed to return to room temperature with stirring and the reaction is continued for 18 hours. During the reaction, the triethylamine hydrochloride formed precipitates in the medium. The solution obtained purified by precipitation is filtered at room temperature and with stirring in 10 litres of absolute ethanol.

The polymer precipitate obtained is recovered and dried under vacuum to constant weight. 145 g of dried polymer are thus obtained.

### EXAMPLE 2

### Waterproof Mascara

The mascara composition below may be prepared:

| | |
|---|---|
| Paraffin wax | 2.2 |
| Carnauba wax | 4.5 |
| Beeswax | 9.9 |
| Modified hectorite (“Bentone 38 V”) from | 5.3 |
| Elementis | |
| Vinyl acetate/allyl stearate copolymer (Mexomer | 2.21 |
| PQ from the company Chimex) | |
| Liposoluble cellulose derivative of Example 1 | 10 AM* |
| Polyvinyl laurate | 0.75 |
| Preserving agents | 0.2 |
| Propylene carbonate | 1.75 |
| Talc | 1 |
| Pigments | 5 |
| Isododecane | qs 100 |

AM*: active material

### EXAMPLE 3

### Wax-in-Water Emulsion Mascara

The mascara composition below may be prepared

| | |
|---|---|
| Candelilla wax | 4.5 |
| Paraffin wax | 4 |
| Beeswax | 8.5 |
| Stearic acid | 5.8 |
| Isononyl isononanoate | 10 |
| Liposoluble derivative of Example 1 | 5 AM |
| Black iron oxide | 8 |
| Triethanolamine | 2.4 |
| Hydroxyethyl cellulose | 0.9 |
| Gum arabic | 3.4 |
| Preserving agents | qs |
| Water | qs 100 |

### EXAMPLE 4

### Liquid Lipstick

| Polymer of Example 1 | 20 AM |
|---|---|
| Isododecane | qs 100 |
| Hydrogenated polybutene | 2.1 |
| Octyldodecanol | 0.9 |
| Phenyl trimethicone DC 556 | 2.1 |
| Vinylpyrrolidone/1-eicosene copolymer (Antaron | 1.2 |
| V 220 ISP) | |
| Pigments | 3 |

### EXAMPLE 5

### Stick of Lipstick

| | |
|---|---|
| Polyethylene wax | 15 |
| Polymer of Example 1 | 15 AM |
| Isododecane | qs 100 |
| Hydrogenated polybutene | 8.2 |
| Octyldodecanol | 3.6 |
| Phenyl trimethicone DC 556 | 4.8 |
| Vinylpyrrolidone/1-eicosene copolymer (Antaron | 1.2 |
| V 220 ISP) | |
| Pigments | 10 |

1. Cosmetic composition for making up the skin, the lips, the eyelashes or the nails, comprising, in a cosmetically acceptable medium, a liquid fatty phase and a liposoluble modified cellulose or cellulose derivative, the said modified cellulose or the said modified cellulose derivative comprising free hydroxyl functions totally or partially replaced with hydrophobic groups chosen from the radicals of formula —OYR, in which:
R represents a group chosen from:
A) hydrocarbon-based groups containing linear or branched, saturated or unsaturated chains, or saturated or unsaturated rings, containing from 8 to 50 carbon atoms for the modified cellulose or from 4 to 50 carbon atoms for the modified cellulose derivative,
the said groups possibly comprising in their chains one or more aromatic groups and/or one or more hetero atoms chosen from O, N, P, Si and S; the said groups possibly being fluorinated or perfluorinated;
B) groups of polymeric nature chosen from polyolefins, hydrogenated or non-hydrogenated polydienes and lipophilic polycondensates, and mixtures thereof,
Y represents a single bond or a divalent bonding group.

2. Composition according to the preceding claim, characterized in that the cellulose derivative is chosen from cellulose esters or ethers.

3. Composition according to claim 1 or 2, characterized in that the cellulose derivative is chosen from cellulose alkyl ethers with an alkyl group containing from 1 to 4 carbon atoms.

4. Cosmetic composition for making up the skin, the lips, the eyelashes or the nails, comprising, in a cosmetically acceptable medium, a liquid fatty phase and a liposoluble modified cellulose ester, comprising free hydroxyl functions totally or partially replaced with hydrophobic groups chosen from the radicals of formula —OYR, in which:
R represents a group chosen from:
A) hydrocarbon-based groups containing linear or branched, saturated or unsaturated chains, or saturated or unsaturated rings, containing from 4 to 50 carbon atoms,
the said groups possibly comprising in their chains one or more aromatic groups and/or one or more hetero atoms chosen from O, N, P, Si and S; the said groups possibly being fluorinated or perfluorinated;
B) groups of polymeric nature chosen from polyolefins, hydrogenated or non-hydrogenated polydienes and lipophilic polycondensates, and mixtures thereof,
Y represents a single bond or a divalent bonding group.

5. Composition according to claim 1 or 2 and 4, characterized in that the cellulose derivative is chosen from esters derived from the reaction of some of the free hydroxyl functions of cellulose with a carboxylic acid or a carboxylic acid derivative containing from 1 to 4 carbon atoms.

6. Composition according to the preceding claim, characterized in that the cellulose esters are chosen from cellulose acetates, propionates, butyrates, isobutyrates, acetobutyrates and acetopropionates, and mixtures thereof.

7. Composition according to one of the preceding claims, in which the divalent bonding group Y is chosen from the groups —(C═O)—, —(C═O)O—, —SO₂—, —CO—NH—, —CO—NR′— and —Si(R₃)₂—, the groups R₃, which may be identical or different, being a linear or branched hydrocarbon-based group containing from 1 to 500 carbon atoms, or a cyclic hydrocarbon-based group containing from 3 to 500 carbon atoms, the said group being saturated or unsaturated and possibly comprising one or more atoms O, N, S, Si and/or P, and R′ denoting an alkyl radical containing from 1 to 4 carbon atoms.

8. Composition according to one of the preceding claims, in which the groups R are chosen from linear-chain hydrocarbon-based groups containing from 8 to 25 carbon atoms for the modified cellulose and linear-chain hydrocarbon-based groups containing from 4 to 25 carbon atoms for the modified cellulose derivatives, in particular saturated linear hydrocarbon-based groups containing from 8 to 11 carbon atoms and linear hydrocarbon-based groups with at least one unsaturation, containing from 8 to 22 carbon atoms.

9. Composition according to the preceding claim, characterized in that the groups R are chosen from saturated linear alkyl groups such as n-butyl, pentyl, n-hexyl, n-heptyl, n-octyl, n-nonyl, n-decyl and n-undecyl, and mixtures thereof.

10. Composition according to one of the preceding claims, characterized in that the groups R are chosen from saturated branched-chain hydrocarbon-based groups containing from 8 to 50 carbon atoms for the modified cellulose and saturated branched-chain hydrocarbon-based groups containing from 4 to 50 carbon atoms for the modified cellulose derivatives.

11. Composition according to claim 10, in which the groups R are chosen from branched alkyl groups containing from 8 to 40 carbon atoms.

12. Composition according to claim 10 or 11, in which the groups R are chosen from isobutyl, tert-butyl,isopentyl, tert-hexyl, 2-ethylhexyl, tert-octyl, isononyl, isodecyl, neodecyl, isododecyl, isohexadecyl and isostearyl groups, and mixtures thereof.

13. Composition according to one of the preceding claims, in which the groups R are chosen from cyclic hydrocarbon-based groups containing from 8 to 50 carbon atoms and preferably from 8 to 20 carbon atoms for the modified cellulose and cyclic hydrocarbon-based groups containing from 6 to 50 carbon atoms and preferably from 6 to 20 carbon atoms for the modified cellulose derivatives.

14. Composition according to claim 13, in which the groups R are chosen from cyclohexyl, isobornyl, adamantyl and norbornyl groups, and mixtures thereof.

15. Composition according to one of the preceding claims, characterized in that the groups R are chosen from branched and/or cyclic hydrocarbon-based groups derived from unsaturated fatty acid derivatives containing from 14 to 22 carbon atoms, such as alkylketene dimers.

16. Composition according to one of claims 1 to 7, characterized in that the polyolefins are chosen from polymers obtained by homopolymerization or copolymerization of monomers chosen from α-olefins containing, for example, from 2 to 20 carbon atoms.

17. Composition according to one of claims 1 to 7, characterized in that the polydienes are chosen from polydienes resulting from the polymerization of dienes containing, for example, from 4 to 20 carbon atoms, such as butadiene, isoprene or hexadiene, or from polymers resulting from the polymerization of dienes containing, for example, from 4 to 20 carbon atoms with other vinyl monomers and/or with styrene or substituted styrenes.

18. Composition according to one of claims 1 to 7, characterized in that the lipophilic polycondensates are chosen from lipophilic polyesters, polyamides, polyester amides, polyurethanes, polycarbonates, polyureas, copolymers (urea/urethane) and polyethers, and mixtures thereof.

19. Composition according to the preceding claim, characterized in that the lipophilic polyesters are derived from the polyesterification of at least one polyol with at least: one polycarboxylic acid, one dicarboxylic or tricarboxylic acid derivative or one alkyl diester containing from 1 to 5 carbon atoms.

20. Composition according to claim 18, characterized in that the polyamides are chosen from the polyamides obtained by condensation between an aliphatic, cycloaliphatic or aromatic dicarboxylic acid (or ester derivative containing from 1 to 4 carbon atoms) containing from 3 to 50 carbon atoms and a linear or branched aliphatic, cycloaliphatic or aromatic diamine containing from 2 to 50 carbon atoms.

21. Composition according to claim 18, characterized in that the polyurethanes, polyureas and polyureas/urethanes are obtained by polyaddition between aliphatic, cycloaliphatic and/or aromatic diisocyanates containing from 4 to 100 carbon atoms and preferably from 4 to 30 carbon atoms and diols or diamines or diol/diamine mixtures.

22. Composition according to one of the preceding claims, characterized in that the group R bears one or more groups capable of establishing a hydrogen bond.

23. Composition according to the preceding claim, in which the group capable of establishing a hydrogen bond is chosen from the groups having the following formulae:
hydroxyl —OH;
carboxylic acid —COOH;
amino-NR₁R₂ with R₁ and R₂ being identical or different;
pyridino of formula: 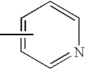
pyrimidino of formula: 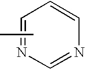
oxazolino corresponding to one of the following formulae: 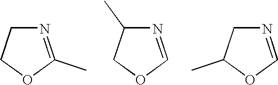
amido of formula —NH—CO—R′ or —CO—NH—R₁;
pyrrolidino corresponding to one of the following formulae: 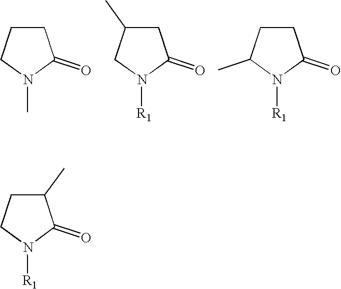
carbamoyl of formula —O—CO—NH—R′ or —NH—CO—O—R′;
thiocarbamoyl of formula —O—CS—NHR₁ or —NH—CS—O—R′;
carbonato —O—CO—O—R′;
ureyl —NR₁—CO—N(R₁)₂, the R₁ being identical or different;
thioureyl —NR₁—CS—N(R₁)₂, the R₁ being identical or different;
oxamido —NR₁—CO—CO—N(R₁)₂ with the R₁ being identical or different;
guanidino —NH—C(═NH)—N(R₁)₂ with the R₁ being identical or different;
biguanidino —NH—C(═NH)—NH—C(=NH)—N(R₁)₂ with the R₁ being identical or different;
sulfonamido —NR₁—S(═O)₂—R′;
with R₁ and R′ representing H or an alkyl group containing from 1 to 4 carbon atoms, R′ representing an alkyl radical containing from 1 to 4 carbon atoms.

24. Composition according to one of the preceding claims, characterized in that the modified cellulose or the modified cellulose derivative is soluble at a concentration of at least 1% by weight relative to the total weight of the composition, in the oil forming the weight majority of the liquid fatty phase, at room temperature (25° C.) and atmospheric pressure (10₅ Pa)

25. Composition according to one of the preceding claims, characterized in that the liposoluble modified cellulose or modified cellulose derivative is film-forming.

26. Composition according to one of the preceding claims, characterized in that the liposoluble modified cellulose or cellulose derivative represents from 0.5% to 50%, preferably from 1% to 45%, better still from 4% to 40% and even better still from 5% to 30% by weight of solids relative to the total weight of the composition according to the invention.

27. Composition according to one of the preceding claims, characterized in that the fatty phase comprises at least one oil chosen from volatile oils.

28. Composition according to the preceding claim, characterized in that the volatile oil is chosen from hydrocarbon-based oils containing from 8 to 16 carbon atoms, linear or cyclic volatile silicone oils especially containing from 2 to 10 silicon atoms, and mixtures thereof.

29. Composition according to claim 27 or 28, characterized in that the volatile oil is chosen from branched C₈-C₁₆ alkanes, for instance C₈-C₁₆ isoparaffins such as isododecane, isodecane and isohexadecane.

30. Composition according to the preceding claim, characterized in that the volatile oil represents from 0.1% to 95% by weight, preferably from 1% to 65% by weight and better still from 2% to 50% by weight relative to the weight of the composition.

31. Composition according to one of the preceding claims, characterized in that the fatty phase comprises at least one non-volatile oil.

32. Composition according to one of the preceding claims, characterized in that the fatty phase represents from 0.01% to 98% by weight, preferably from 0.05% to 75% by weight and better still from 1% to 60% by weight relative to the total weight of the composition.

33. Composition according to one of the preceding claims, characterized in that the fatty phase comprises at least one non-volatile oil.

34. Composition according to one of the preceding claims, characterized in that it comprises an aqueous phase.

35. Composition according to the preceding claim, characterized in that the aqueous phase represents from 0.1% to 65% by weight, preferably from 1% to 55% by weight and better still from 5% to 50% by weight relative to the total weight of the composition.

36. Composition according to one of claims 1 to 34, characterized in that it is anhydrous.

37. Composition according to one of the preceding claims, characterized in that it comprises an additional film-forming polymer chosen from synthetic polymers, of free-radical type or of polycondensate type, and polymers of natural origin, and mixtures thereof.

38. Composition according to the preceding claim, characterized in that the additional film-forming polymer is chosen from acrylic polymers, polyurethanes, polyesters, polyamides, polyureas, cellulose polymers other than the liposoluble modified cellulose derivatives, and mixtures thereof.

39. Composition according to the preceding claim, characterized in that the additional film-forming polymer represents from 0.1% to 30% by weight and better still from 0.5% to 15% by weight of solids relative to the total weight of the composition.

40. Cosmetic composition according to any one of the preceding claims, characterized in that it also comprises one or more dyestuffs chosen from water-soluble dyes and pulverulent dyestuffs, such as pigments, nacres and flakes.

41. Composition according to the preceding claim, characterized in that the dyestuff is present in a content ranging from 0.01% to 50% by weight and preferably from 0.01% to 30% by weight relative to the weight of the composition.

42. Composition according to any one of the preceding claims, characterized in that it comprises at least one fatty substance that is solid at room temperature, chosen from waxes, pasty fatty substances and gums, and mixtures thereof.

43. Composition according to any one of the preceding claims, characterized in that it contains from 0.1% to 50%, better still from 1% to 40% and even better still from 5% to 20% by weight of waxes relative to the total weight of the composition.

44. Composition according to one of the preceding claims, characterized in that it comprises a filler.

45. Composition according to the preceding claim, characterized in that the filler is present in a content ranging from 0.01% to 50% by weight and preferably ranging from 0.01% to 30% by weight relative to the total weight of the composition.

46. Composition according to one of the preceding claims, characterized in that it comprises a lipophilic or hydrophilic, organic or mineral, polymeric or molecular gelling agent.

47. Composition according to the preceding claim, characterized in that the lipophilic or hydrophilic gelling agent is present in a content ranging from 0.05% to 40% by weight, preferably from 0.5% to 20% and better still from 1% to 15% by weight relative to the total weight of the composition.

48. Composition according to any one of the preceding claims, characterized in that it comprises a cosmetic ingredient chosen from vitamins, thickeners, gelling agents, trace elements, softeners, sequestrants, fragrances, acidifying or basifying agents, preserving agents, sunscreens, surfactants, antioxidants, fibres, hair loss counteractants, eyelash care agents, antidandruff agents and propellants, or mixtures thereof.

49. Cosmetic composition according to any one of the preceding claims, characterized in that it is in form of a suspension, a dispersion, a solution, a gel, an emulsion, especially an oil-in-water (O/W) or water-in-oil (W/O) emulsion, or a multiple emulsion (W/O/W, polyol/O/W or O/W/O), or in the form of a cream, a paste, a mousse, a dispersion of vesicles, especially of ionic or nonionic lipids, a two-phase or multi-phase lotion, a spray, a powder, a paste, especially a soft paste or an anhydrous paste, a stick or a cast solid.

50. Composition according to one of the preceding claims, characterized in that it is a makeup product for keratin fibres.

51. Composition according to one of the preceding claims, characterized in that it is a mascara.

52. Composition according to one of claims 1 to 49, characterized in that it is a skin makeup product.

53. Composition according to one of claims 1 to 49, characterized in that it is a lip makeup product.

54. Use of a composition according to one of claims 1 to 53 for improving the resistance and/or the transfer resistance of the makeup on keratin materials.

55. Use of a liposoluble modified cellulose or cellulose derivative, the said modified cellulose or the said modified cellulose derivative comprising free hydroxyl functions totally or partially replaced with hydrophobic groups chosen from the radicals of formula —OYR, in which:
R represents a group chosen from:
A) hydrocarbon-based groups containing linear or branched, saturated or unsaturated chains, or saturated or unsaturated rings, containing from 8 to 50 carbon atoms for the modified cellulose or from 4 to 50 carbon atoms for the modified cellulose derivative,
the said groups possibly comprising in their chains one or more aromatic groups and/or one or more hetero atoms chosen from O, N, P, Si and S; the said groups possibly being fluorinated or perfluorinated;
B) groups of polymeric nature chosen from polyolefins, hydrogenated or non-hydrogenated polydienes and lipophilic polycondensates, and mixtures thereof,
Y represents a single bond or a divalent bonding group, to obtain a composition that has a good texture, that is easy to apply and that gives on the skin, the lips or keratin fibres a deposit that shows good resistance and/or that does not transfer.

56. Cosmetic process for making up keratin materials, which consists in applying to the said keratin materials a cosmetic composition comprising, in a cosmetically acceptable medium, a liquid fatty phase and a liposoluble modified cellulose or cellulose derivative, the said modified cellulose or the said modified cellulose derivative comprising free hydroxyl functions totally or partially replaced with hydrophobic groups chosen from the radicals of formula —OYR, in which:
R represents a group chosen from:
A) hydrocarbon-based groups containing linear or branched, saturated or unsaturated chains, or saturated or unsaturated rings, containing from 8 to 50 carbon atoms for the modified cellulose or from 4 to 50 carbon atoms for the modified cellulose derivative,
the said groups possibly comprising in their chains one or more aromatic groups and/or one or more hetero atoms chosen from O, N, P, Si and S; the said groups possibly being fluorinated or perfluorinated;
B) groups of polymeric nature chosen from polyolefins, hydrogenated or non-hydrogenated polydienes and lipophilic polycondensates, and mixtures thereof,
Y represents a single bond or a divalent bonding group.

57. Cosmetic process for making up keratin materials, which consists in applying to the said keratin materials a cosmetic composition comprising, in a cosmetically acceptable medium, a liquid fatty phase and a liposoluble modified cellulose ester, the said modified cellulose ester comprising free hydroxyl functions totally or partially replaced with hydrophobic groups chosen from the radicals of formula —OYR, in which:
R represents a group chosen from:
A) hydrocarbon-based groups containing linear or branched, saturated or unsaturated chains, or saturated or unsaturated rings, containing from 4 to 50 carbon atoms, the said groups possibly comprising in their chains one or more aromatic groups and/or one or more hetero atoms chosen from O, N, P, Si and S;
the said groups possibly being fluorinated or perfluorinated;
B) groups of polymeric nature chosen from polyolefins, hydrogenated or non-hydrogenated polydienes and lipophilic polycondensates, and mixtures thereof,
Y represents a single bond or a divalent bonding group.

58. Cosmetic composition comprising, in a cosmetically acceptable medium, a liquid fatty phase and a liposoluble modified cellulose or cellulose ester, the said modified cellulose or the said modified cellulose ester comprising free hydroxyl functions totally or partially replaced with hydrophobic groups chosen from the radicals of formula —OYR, in which:
R represents a group chosen from:
A) hydrocarbon-based groups containing linear or branched, saturated or unsaturated chains, or saturated or unsaturated rings, containing from 8 to 50 carbon atoms for the modified cellulose or from 4 to 50 carbon atoms for the modified cellulose ester, the said groups possibly comprising in their chains one or more aromatic groups and/or one or more hetero atoms chosen from O, N, P, Si and S; the said groups possibly being fluorinated or perfluorinated;
B) groups of polymeric nature chosen from polyolefins, hydrogenated or non-hydrogenated polydienes and lipophilic polycondensates, and mixtures thereof,
Y represents a single bond or a divalent bonding group.

59. Anhydrous cosmetic composition comprising, in a cosmetically acceptable medium, a liquid fatty phase and at least 4% of a liposoluble modified cellulose derivative, the said modified cellulose derivative comprising free hydroxyl functions totally or partially replaced with hydrophobic groups chosen from the radicals of formula —OYR, in which:
R represents a group chosen from:
A) hydrocarbon-based groups containing linear or branched, saturated or unsaturated chains, or saturated or unsaturated rings, containing from 4 to 50 carbon atoms,
the said groups possibly comprising in their chains one or more aromatic groups and/or one or more hetero atoms chosen from O, N, P, Si and S; the said groups possibly being fluorinated or perfluorinated;
B) groups of polymeric nature chosen from polyolefins, hydrogenated or non-hydrogenated polydienes and lipophilic polycondensates, and mixtures thereof,
Y represents a single bond or a divalent bonding group.
